(19) United States
(12) Patent Application Publication
(10) Pub. No.: US 2020/0246621 A1
(43) Pub. Date: Aug. 6, 2020

(54) SYSTEMS AND METHODS FOR PAIN MANAGEMENT

(71) Applicant: ANCORA MEDICAL, INC., Menlo Park, CA (US)

(72) Inventors: Jonathan B. Hofius, La Honda, CA (US); Manish Kothari, San Carlos, CA (US); Chunyuan Qiu, Huntington Beach, CA (US)

(21) Appl. No.: 16/779,368

(22) Filed: Jan. 31, 2020

Related U.S. Application Data
(60) Provisional application No. 62/799,638, filed on Jan. 31, 2019.

(51) Int. Cl. A61N 1/36; A61M 25/00; G16H 20/10; G16H 20/40

(52) U.S. Cl. CPC A61N 1/36071; A61M 25/0043; A61M 2025/0063; G16H 20/40; G16H 20/10

(57) ABSTRACT

Methods and systems are provided for a dual-action catheter. In one example, a dual-action catheter system comprises a catheter including a catheter lumen, a bioelectric neuromodulation stylet of a diameter smaller than the lumen of the catheter for insertion of the bioelectric neuromodulation stylet within the catheter lumen, one or more electrodes positioned at a tip end of the bioelectric neuromodulation stylet, and a delivery pathway for delivery of pharmacological treatments therethrough while the bioelectric neuromodulation stylet is inserted within the catheter lumen.

### CROSS REFERENCE TO RELATED APPLICATIONS

The present application claims priority to U.S. Provisional Application No. 62/799,638, entitled “SYSTEMS AND METHODS FOR PAIN MANAGEMENT”, and filed on Jan. 31, 2019. The entire contents of the above-identified application are hereby incorporated by reference for all purposes.

### FIELD OF THE INVENTION

The present disclosure pertains generally to managing pain in human patients. The managing of pain is via a dual-action catheter system configured to selectively deliver pharmacological and bioelectrical neuromodulation as a function of one or more variables related to pain management.

### BACKGROUND OF THE INVENTION

Currently over two hundred million major surgeries are performed each year. During and/or following such surgeries, it is common for patients to experience moderate to severe pain. To address such issues related to pain, physicians employ use of pharmacological agents and aggressive pain management protocols that can include one or more of narcotics, non-steroidal anti-inflammatory drugs (NSAIDs), anticonvulsants, and antidepressants. However, despite widespread use of such strategies, post-operative pain remains insufficiently controlled. Complications related to pain can in some examples lead to poor clinical outcomes, including but not limited to deep vein thrombosis (DVT), pulmonary embolism (PE), post-operative myocardial infarction, and pneumonia. Post-operative pain can also prolong a patient's hospital stay, can in some cases lead to emergency room visits and/or hospital readmission, and may result in overall decreased patient satisfaction. Further complications of post-operative pain can include adverse effects on patient immune system function, predisposition of the patient to an increased risk of opioid abuse, and development of chronic pain syndrome.

Post-operative pain and discomfort can in some examples (e.g. breast surgery, hernia surgery, total knee replacement, thoracotomies) last for weeks to months. For such patients, current treatment options are limited beyond the use of narcotics, and there are well-known tolerance issues and dependency issues associated with prolonged use of narcotics. Various attempts have been made in the past to address such issues, and recent advancements in cryotherapy and peripheral nerve activity modulation have indicated potential for alleviating prolonged sub-chronic post-operative pain. However, while both of the above-mentioned methodologies represent promising avenues for pain management, their potential for clinical application is currently largely unknown.

A synergetic effect of pharmacological and electrical neuromodulation strategies at a peripheral nerve location may have potential advantages over either pharmacological or electrical treatment alone for perioperative and/or postoperative pain because of the dynamic nature of the pain. The potential advantages may relate in particular to the omission or reduction of agents which may result in side effects. For example, pharmacological pain managements can be especially effective at managing severe pain, but the benefits may be short-lived due to drug tolerance issues and side effects. Alternatively, electrical nerve modulation strategies as discussed above can be less effective for management of severe pain, but may be effective for management of minor to moderate pain without the undesirable consequences of medications. As pain, especially pain during and/or following surgery and/or trauma, is a dynamic phenomenon where the onset, intensity and duration are subject to changes in response to one or more of physiological, pathological and psychological conditions of patients, a pain therapy tailored toward the needs of particular patients, is needed. The inventors have herein recognized such issues, and have developed systems and methods for effective pain management that takes into account the benefits and drawbacks of the above-mentioned pain management strategies.

### SUMMARY OF THE INVENTION

Provided herein are systems and methods related to dual-action catheter systems that target sensorial afferent pain pathway(s) (and avoidance of motor pathways), where the dual-action aspect relates to an ability of the catheter system to deliver electrical treatments, also referred to herein as bioelectrical neuromodulation or neuromodulation treatments, and/or pharmacological treatments (which may be understood to include a form of neuromodulation as well) to a desired tissue site of a patient. The systems and methods herein disclosed relate to extravascular implantation of the disclosed catheter systems.

More specifically, bioelectric neuromodulation as discussed herein encompasses one or more of the following examples. It may be understood that, with regard to the following examples, there may be some overlap between examples. In a first example, bioelectric neuromodulation may comprise electrical nerve block, which may include electrically-induced blocking of the activation of small sensorial nerve fibers such as A-beta fibers, C-fibers, and A-delta fibers. Said another way, electrical nerve block may comprise the stopping of sensorial input (e.g. a pain signal) from being transmitted to the spinal cord and the brain.

In a second related example, activation or stimulation of certain large nerve fibers may inhibit small nerve fiber (e.g. A-beta, C-fibers, and A-delta fibers) transmission, thus leading to a reduced pain response. Such methodology is based on “gate control theory.”

A third example of bioelectric neuromodulation, as discussed herein, relates to the alteration of nerve activity through a targeted delivery of a stimulus, the stimulus comprising electrical stimulation or delivery of chemical agent(s), to specific neurological sites in a human body. In terms of electrical stimulation, there may be many variations of an electrical stimulation pattern that are encompassed as defined herein as bioelectric neuromodulation. Examples include frequency, amplitude, applied current and duration, monophasic stimulation, biphasic stimulation, synchronized and/or unsynchronized stimulation in terms of a sensed signal, variable stimulation (e.g. one or more of variable amplitude, variable frequency, variable current and duration, etc.). Variations in terms of electrical stimulation may enable differential stimulation, for example not only in terms of targeting sensorial nerves vs. motor nerves, but also in terms of selectively targeting different nerve fibers including but not limited to the A-beta, C-fibers and A-delta fibers mentioned above. Discussed herein, it may be further understood that bioelectric neuromodulation may further include electrical nerve modulation strategies which result in hormone/ligand effects (e.g. endorphins, inflammatory mediators, etc.) which are not limited to pain, per say, but also to other neuromodulatory effects potentially beneficial for use in terms of a wide range of diseases including but not limited to Parkinson's disease, depression, sleep apnea, etc.

In one example, the dual-action catheter system(s) of the present disclosure include a lumen that is capable of receiving a stylet therethrough, the stylet capable of sensing/transmitting electrical signals and/or providing electrical stimulation. Discussed herein, such a stylet is referred to as a bioelectric neuromodulation stylet, or more simply as a stylet. Specifically, the bioelectric neuromodulation stylet may be capable of conducting electricity and thus may be configured to deliver electrical pulses to a desired tissue site, and may additionally or alternatively be configured to transmit electrical signals recorded from a particular tissue site. Said another way, the bioelectric neuromodulation stylet may comprise a bi-directional sensing and stimulating neuromodulation stylet.

The bioelectric neuromodulation stylet may be inserted into the lumen of the catheter while the catheter is implanted and/or removed from the lumen of the catheter while the catheter is implanted. In this way, the bioelectric neuromodulation stylet may be inserted into an implanted catheter either prior to a surgical procedure on the patient for which the catheter is implanted, during the surgical procedure, or any time after the surgery. Stimulation via the bioelectric neuromodulation stylet may thus be utilized as a means for one or more of providing bioelectric neuromodulation prior to initiation of the surgical procedure (e.g. preemptive analgesia), providing bioelectric neuromodulation during the surgical operation (e.g. utilizing bioelectric neuromodulation strategies for analgesia purposes during surgery), and/or for providing bioelectric neuromodulation after the surgical operation (e.g. for analgesic purposes and/or promoting desired functional responses). Thus, it may be understood that discussed herein, neuromodulation via the stylet encompasses pain treatment (e.g. analgesia) as well as the promotion of desired functional effects. It may be understood that such desired functional effects may be related to reducing undesirable effects related to one or more of Parkinson's disease, seizures, female incontinence, sleep apnea, depression, etc.

In some examples the bioelectric neuromodulation stylet may be hollow, where pharmacological treatments may be delivered therethrough, or may be solid. In a case where the bioelectric neuromodulation stylet is solid, pharmacological treatments may be delivered to a desired tissue site via a space or spaces between the bioelectric neuromodulation stylet and internal walls of the catheter. In some examples, the bioelectric neuromodulation stylet may protrude a distal end of the catheter, whereas in other examples the bioelectric neuromodulation stylet may be flush with the distal end of the catheter. The bioelectric neuromodulation stylet may conduct electricity at a distal tip of the stylet, where the remaining portions of the bioelectric neuromodulation stylet are electrically isolated or insulated. However, in other examples where the bioelectric neuromodulation stylet conducts electricity at the distal tip, the remaining portions of the bioelectric neuromodulation stylet may also be capable of conducting electricity, without departing from the scope of the present disclosure. The bioelectric neuromodulation stylet may have a determined rigidity for ease of insertion into the lumen of the catheter while the catheter is implanted, and may additionally have a particular flexibility to accommodate body movement. In some examples, the bioelectric neuromodulation stylet may be coiled, which may serve to increase flexibility and/or kink resistance.

In some examples, the dual-action catheter system of the present disclosure may include a catheter with an inner and an outer sheath that move relative to one another and where a portion of the outer sheath reversibly forms a tissue lock or anchor as a function of the movement of the outer sheath relative to the inner sheath. In such an example, a coil may be positioned within the inner sheath, the coil being capable of conducting electricity and/or capable of being used for catheter location via echolocation strategies. However, in other examples the coil may not be capable of conducting electricity and/or for being used for catheter location, but rather may serve to function as a means of increased flexibility and/or kink resistance, without departing from the scope of this disclosure. In such an example where the catheter includes the inner sheath, the inner sheath of the catheter may define a lumen which may receive the neuromodulation stylet as discussed above.

While in some examples a relative movement of the inner and outer sheath with respect to one another may form the reversible tissue anchor, there may be other variations of such a tissue anchor included for catheter systems of the present disclosure. Examples may include catheters with an inflatable balloon near the tip of the catheter, catheters with one or more barbs near the tip of the catheter, catheters with a hook at or near the tip, etc. Regardless of the exact type of tissue anchor, it may be understood that the basic concept of such an anchor is that the anchor is formed by an increase in diameter (e.g. malecot, balloon, etc.) of the catheter, an increased resistance to movement of the catheter (e.g. barbed, hook, protrusion), etc.

In some examples, the tip of the bioelectric neuromodulation stylet may be configured so as to provide a tissue anchor itself. Such examples may apply, in particular, to bioelectric neuromodulation stylets which extend beyond a tip of the catheter (as opposed to a case where the neuromodulation stylet is flush with the tip of the catheter). Similar to that disclosed above for tissue anchors for the catheter itself, a tissue anchor of a bioelectric neuromodulation stylet may comprise a stylet capable of increasing its diameter (e.g. balloon or malecot) at or near a tip of the neuromodulation stylet, a stylet that includes a capability for an increased resistance to movement (e.g. barbed), or in some examples a glue positioned at or near the tip of the stylet.

The catheter systems and associated neuromodulation stylets of the present disclosure may optionally be coupled to a decoupling system, also referred to herein as a decoupler or decoupler system. Such a decoupler may allow for a limited range of motion for a proximal end of the catheter and/or bioelectric neuromodulation stylet (e.g. end opposite a tip of the catheter and/or tip of the neuromodulation stylet). Such a decoupler may be coupled or attached temporarily to the skin at or near a catheter exit site, and may limit movement of the catheter and/or bioelectric neuromodulation stylet in response to patient movement or external forces such as forces which may pull upon the catheter and/or bioelectric neuromodulation stylet.

Thus, discussed herein, in one example a catheter system of the present disclosure may be mechanically coupled to a decoupler, and may not include a tissue anchor associated with the catheter itself or the neuromodulation stylet inserted therethrough. In another example, a catheter system of the present disclosure may include a tissue anchor (e.g. associated with the catheter and/or the neuromodulation stylet), but may not include coupling the catheter to a decoupler. In still another example, a catheter system of the present disclosure may include a tissue anchor (e.g. associated with the catheter and/or the neuromodulation stylet), where such a catheter system is further coupled to a decoupler. A still further example includes a catheter system without a tissue anchor and where the catheter system is not coupled to a decoupler.

For any of the above-examples encompassed by the present disclosure, bioelectric neuromodulation may be provided via a battery-operated or an electrical outlet powered sensing/stimulating source. The sensing/stimulating source may control a frequency, amplitude, duration, etc., of bioelectric neuromodulation delivered to a desired tissue site. In some examples, a patient may control such parameters, while in other examples, a physician or other user may control such parameters. In still other examples, bioelectric neuromodulation and/or pharmacological treatments may be automated, or at least partially automated. More specifically, total automation may rely on the sensing mechanism of the neuromodulation stylet, including but not limited to one or more of evoked potentials or patterns of evoked potentials from a nerve sensed by the sensing mechanism, firing pattern of C-fibers, A-beta fibers and/or A-delta fibers, etc. In response to particular evoked potentials and/or particular firing patterns, appropriate bioelectric neuromodulatory and/or pharmacological treatments may be automatically delivered to the patient.

On the other hand, partial automation may comprise patient-controlled analgesia, based at least in part on patient pain level as defined by the patient. As examples, a patient may enter a particular pain level they are experiencing into a pain management application (e.g. software application) that is in turn communicatively coupled to a controller that schedules the bioelectric neuromodulation and/or pharmacological treatments. In some examples, partial automation may also rely on information related to one or more of sensed evoked potentials and/or firing patterns of C-fibers, A-beta fibers and/or A-delta fibers. For example, such a system may rely on providing particular bioelectrical neuromodulation and/or pharmacological treatments based on information conveyed via the sensing mechanism of the bioelectric neuromodulation stylet, but which may be altered as a function of pain level as input via the patient. For example, an amount/pattern of bioelectric neuromodulation and/or pharmacological treatment may be automatically provided to the patient, and under conditions where a pain level being experienced by the patient exceeds a capability of the particular amount/pattern of bioelectrical neuromodulation treatment and/or pharmacological treatment, then additional bioelectric neuromodulation and/or pharmacological treatment may be provided as compensation, provided such additional treatments are allowed/approved as will be discussed below. Alternatively, in a case where a particular amount/pattern of bioelectrical neuromodulation and/or pharmacological treatments are automatically being provided to the patient and the patient inputs a lower level of pain than that which the current amount/pattern of electrical treatment and/or pharmacological treatment is inferred to address, then the current amount/pattern of bioelectric neuromodulation and/or pharmacological treatment may be correspondingly reduced.

Thus, for the reduction of pre-operative, intraoperative (also referred to herein as perioperative), and/or post-operative pain in patients, the systems and methods discussed herein may enable a combination of bioelectric neuromodulation and/or pharmacological treatments. The bioelectric neuromodulation and/or pharmacological treatments may in some examples be administered simultaneously, sequentially, automatically, via partial automation, manually, and/or or on-demand.

In some examples, variables related to at least frequency, intensity and/or duration (among other variables discussed above) of bioelectric neuromodulation and/or pharmacological treatments may be recorded via a controller that stores instructions for delivering such bioelectric neuromodulation and/or pharmacological treatments. Furthermore, via the sensing capability of the neuromodulation stylet of the present disclosure, various data related to sensed neural activity in the vicinity of the neuromodulation stylet as a function of the bioelectric neuromodulation and/or pharmacological treatments may be recorded and stored at the controller. Such data may relate to effectiveness of particular bioelectric neuromodulatory and/or pharmacological treatments (for example in terms of pain response). Such data may be uploaded from the controller to one or more server(s) (e.g. local server, remote server, cloud-based server) via existing wired or wireless network(s).

Further data which may be obtained and uploaded to the one or more servers may include patient-inputted responses (e.g. in terms of pain) as a function of particular bioelectric neuromodulation and/or pharmacological treatments. In one example, such responses may be input into a pain management application via the patient themselves. In another example, such responses may be input into the pain management application by a technician or physician, where such responses are communicated (e.g. verbally, written communication, etc.) to the technician or physician by the patient.

Still further data which may be obtained and uploaded to the one or more servers may include health-related data for patients whose data regarding pain management is also uploaded to the one or more servers. Specifically, patient data including one or more of healthcare history (e.g. electronic medical health records), personal history, genomics data, epigenomics data, proteomics data, etc., may be uploaded to the one or more servers.

An analytics module which may access the data stored at the one or more servers via the existing wired or wireless networks may include instructions for performing data analysis on the data. The data analysis may allow for optimizing any number of parameters related to the providing of bioelectric neuromodulation and/or pharmacological treatments to patients. As an example, the platform may conduct one or more of machine learning operations, predictive analytics, deep learning, etc., on the data, such that optimal parameters for providing bioelectric neuromodulation and/or pharmacological treatments may be learned for individual patients and/or similarly situated (in terms of one or more of medical history, genetic background, disease states, etc.) groups of patients, as will be discussed in further detail below.

In this way, issues related to overuse including but not limited to tolerance and/or dependency, may be avoided or reduced, while achieving a desired reduction in pain and/or desired functional effects for particular patient(s). As an example, the systems and methods discussed herein with regard to at least partially patient-controlled pain management or analgesia may include one or more thresholds for bioelectric neuromodulation and/or pharmacological treatments, such that the patient cannot exceed such thresholds, thereby reducing potential for issues related to overuse. Such thresholds may be set/updated in some examples as a function of optimal parameters for bioelectric neuromodulation and/or pharmacological treatments learned via the analytics module discussed above. Such thresholds may be automatically updated in some examples, while in other examples the thresholds may use inputs from a provider or administrator (e.g. physician, nurse, technician, etc.). While the thresholds discussed above may in some examples be based on data learned via the analytics module discussed above, in other examples the thresholds may be set by an administrator without relying on learned data. For example, administrator intervention in terms of setting thresholds may be utilized as a function of a particular patients' clinical situation such as when the patient is known to be a chronic abuser of certain drugs, or in a case where a patient is experiencing end-stage cancer pain, etc. In some examples, the administrator may set thresholds based on a combination of learned data and information obtained the patient (e.g. verbally, orally, written communication, medical history, lab results, etc.).

### BRIEF DESCRIPTION OF THE FIGURES

FIG. 1A depicts an example pain management system of the present disclosure.

FIGS. 1B-1C depict example illustrations of a catheter system that includes a removable bioelectric neuromodulation stylet.

FIG. 1D depicts a more detailed view of select aspects of the pain management system of FIG. 1A.

FIGS. 2A-2B depict examples illustrations of the catheter system of FIGS. 1B-1C, illustrating capabilities for providing pharmacological treatments to desired tissue site(s) via the catheter systems of the present disclosure.

FIG. 2C depicts an example illustration of a decoupler that can be mechanically coupled to a catheter or a bioelectric neuromodulation stylet of the present disclosure.

FIG. 2D depicts a chart showing a variety of combinations related to decoupler and tissue anchor(s) for catheter systems of the present disclosure.

FIGS. 3A-3B depict close-up views of the bioelectric neuromodulation stylet of FIGS. 1B-2B.

FIG. 4A depicts an example illustration of a catheter system of the present disclosure.

FIG. 4B depicts an example embodiment of a bioelectric neuromodulation stylet of the present disclosure.

FIG. 4C depicts another example embodiment of a bioelectric neuromodulation stylet of the present disclosure.

FIG. 5 depicts an example of a catheter of the present disclosure, including a needle through the catheter and a catheter lip.

FIG. 6 depicts a high-level example method for implanting/using the dual-action catheter systems of the present disclosure.

FIG. 7 depicts a high-level example method for providing pain management to a patient, according to the present disclosure.

FIG. 8 depicts an example timeline for providing pain management to a patient, according to the present disclosure.

### DETAILED DESCRIPTION OF THE INVENTION

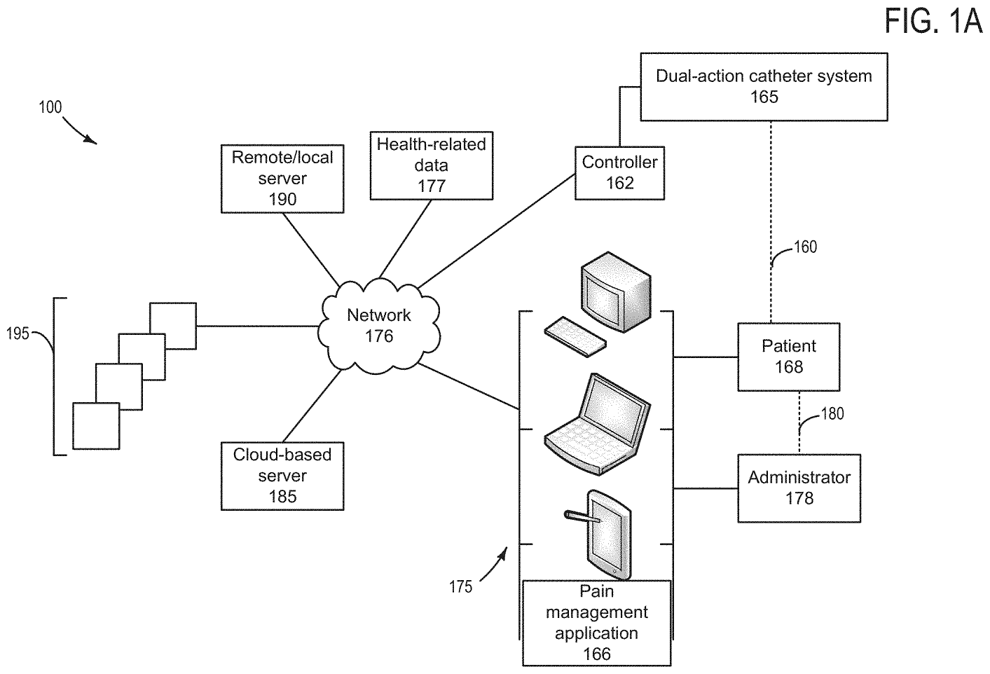

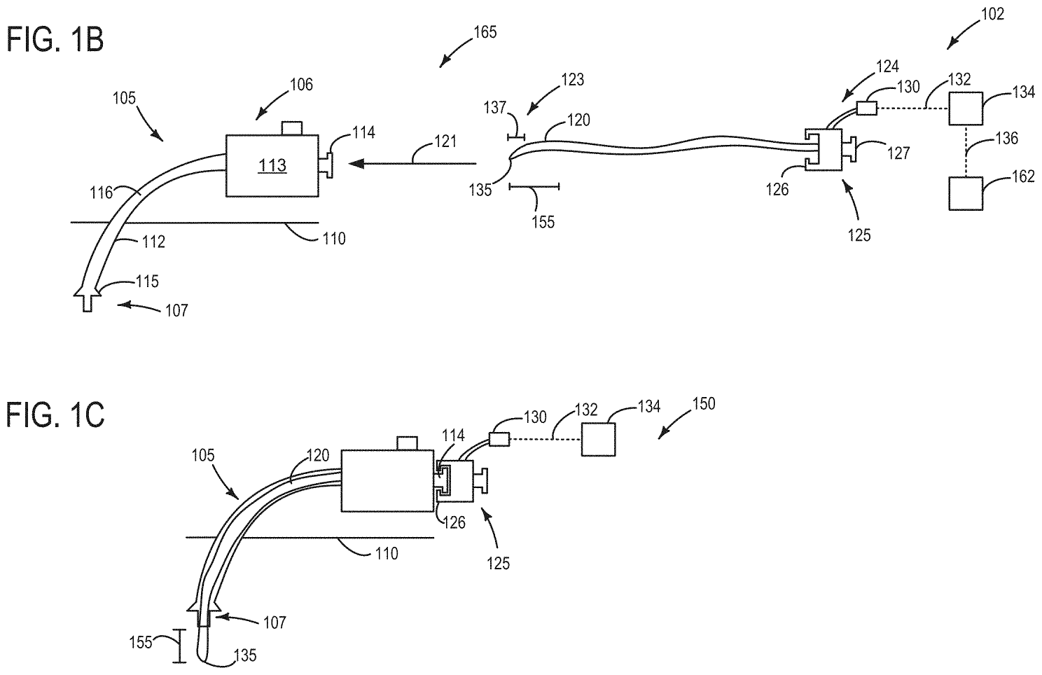

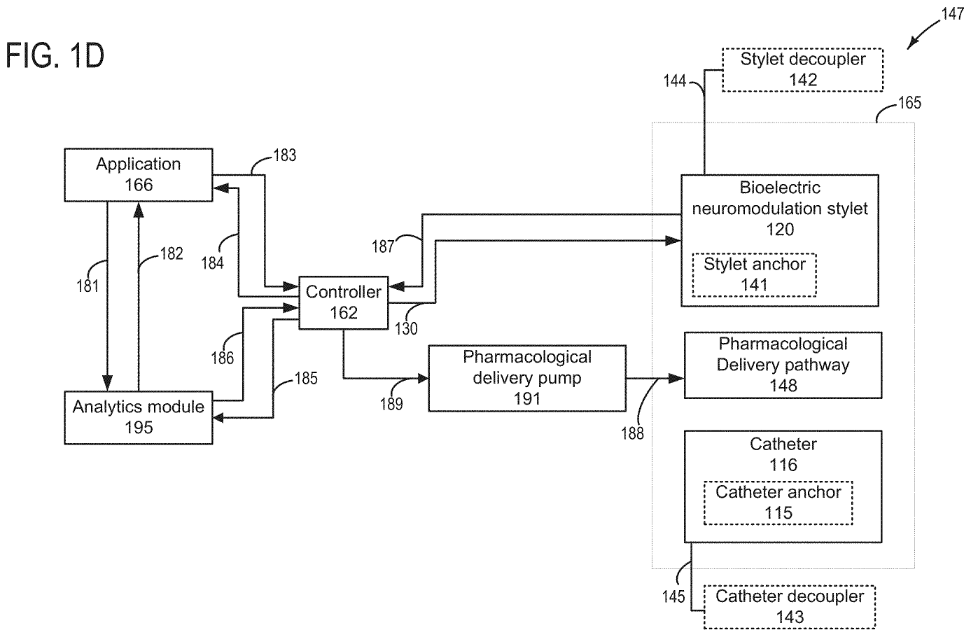

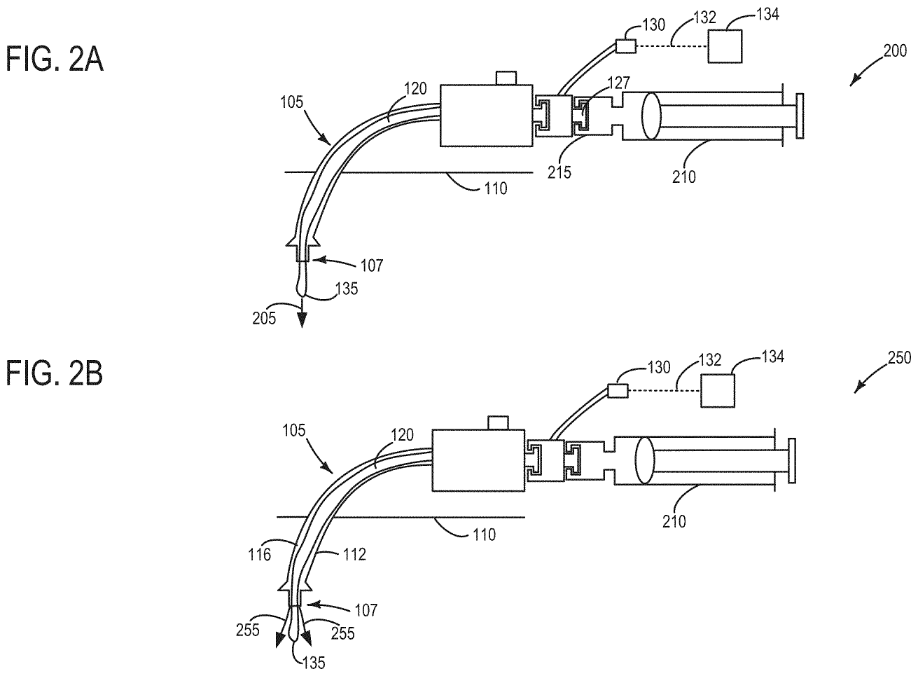

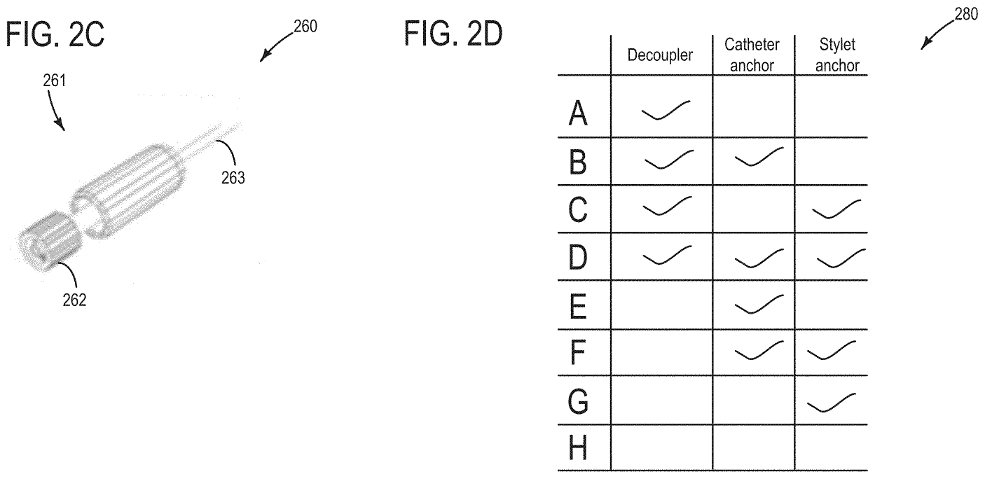

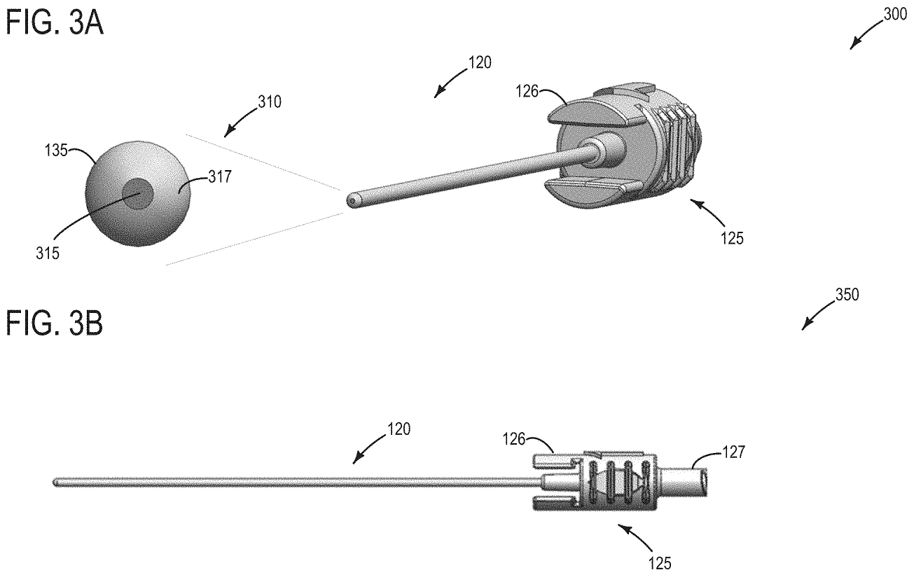

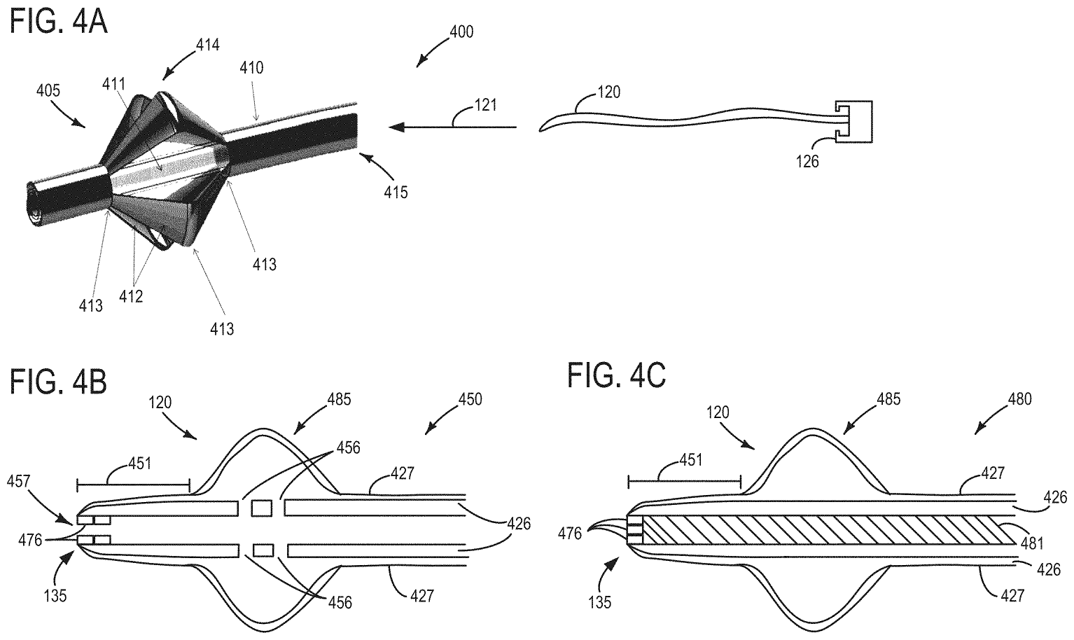

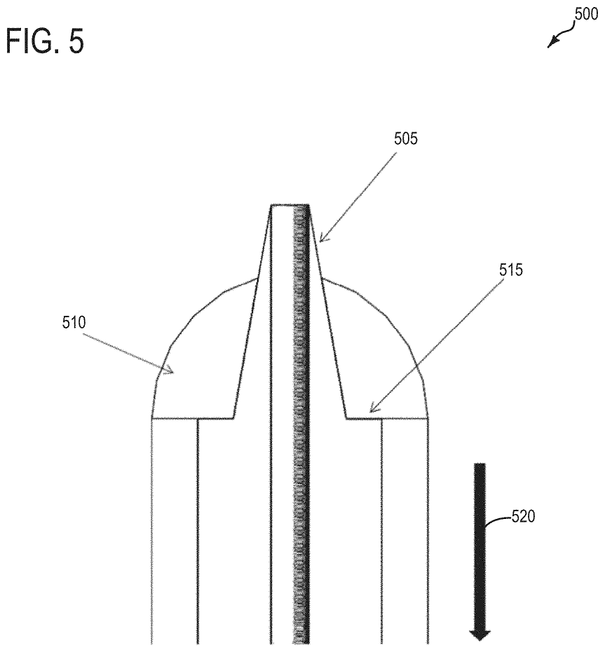

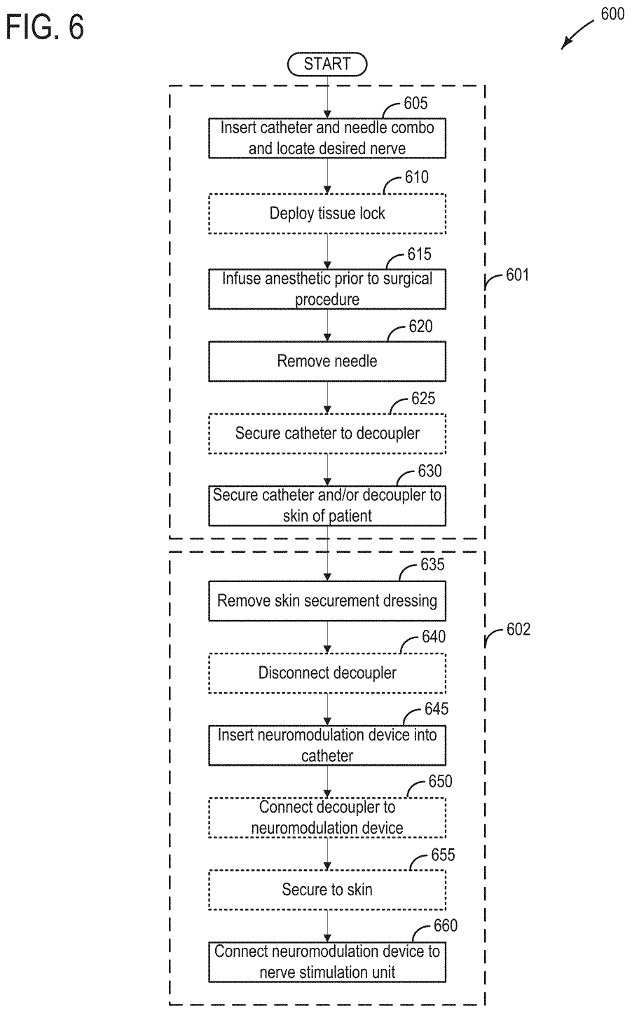

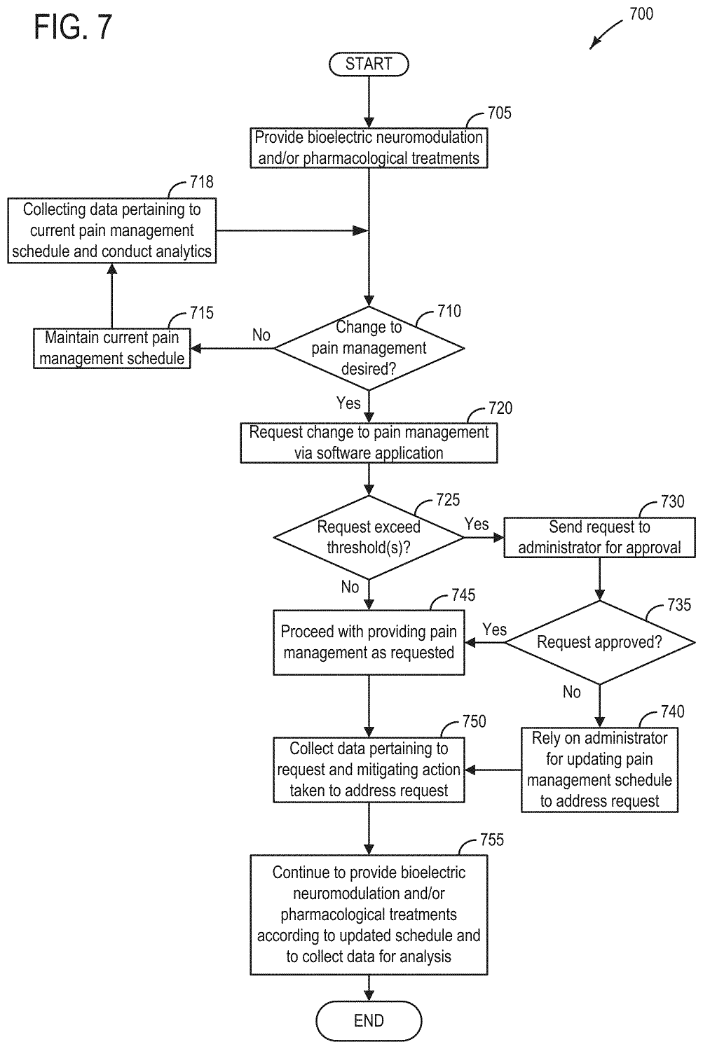

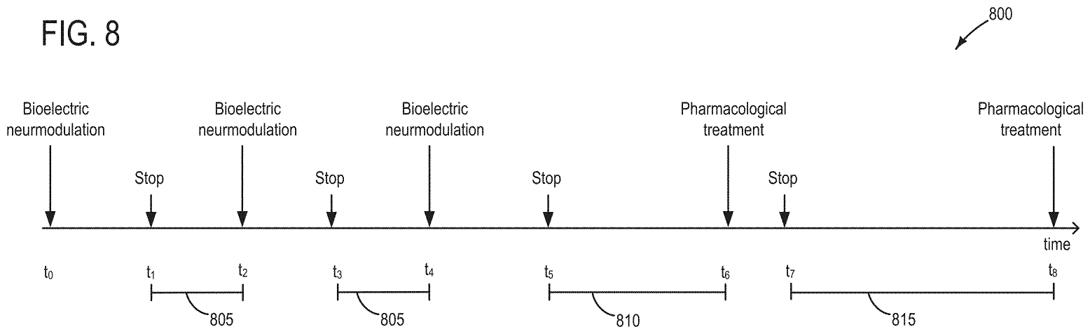

The following description relates to systems and methods for a dual-action catheter. FIG. 1A depicts an example pain management system of the present disclosure. FIGS. 1B-1C depict example illustrations of a catheter system that includes a removable bioelectric neuromodulation stylet. FIG. 1D depicts a more detailed view of select aspects of the pain management system of FIG. 1A. FIGS. 2A-2B depict examples illustrations of the catheter system of FIGS. 1B-1C, illustrating capabilities for providing pharmacological treatments to desired tissue site(s) via the catheter systems of the present disclosure. FIG. 2C depicts an example illustration of a decoupler that can be mechanically coupled to a catheter or a bioelectric neuromodulation stylet of the present disclosure. FIG. 2D depicts a chart showing a variety of combinations related to decoupler and tissue anchor(s) for catheter systems of the present disclosure. FIGS. 3A-3B depict close-up views of the bioelectric neuromodulation stylet of FIGS. 1B-2B. FIG. 4A depicts an example illustration of a catheter system of the present disclosure. FIG. 4B depicts an example embodiment of a bioelectric neuromodulation stylet of the present disclosure. FIG. 4C depicts another example embodiment of a bioelectric neuromodulation stylet of the present disclosure. FIG. 5 depicts an example of a catheter of the present disclosure, including a needle through the catheter and a catheter lip. FIG. 6 depicts a high-level example method for implanting/using the dual-action catheter systems of the present disclosure. FIG. 7 depicts a high-level example method for providing pain management to a patient, according to the present disclosure. FIG. 8 depicts an example timeline for providing pain management to a patient, according to the present disclosure.

Turning to FIG. 1A, depicted is an example illustration of pain management system 100 of the present disclosure. Dual-action catheter system 165, as discussed above, includes at least a catheter for insertion/implantation into a patient, the catheter including at least a lumen which may receive a removable bioelectric neuromodulation stylet. The dual action catheter system may include a reversible tissue anchor at or near (e.g. within 1-10 mm) a tip of the catheter. In other examples, the dual-action catheter system may additionally or alternatively include a reversible tissue anchor at or near (e.g. within 1-10 mm) the tip of the bioelectric neuromodulation stylet, for situations where the bioelectric neuromodulation stylet protrudes from the tip of the catheter. In some examples both catheter and the bioelectric neuromodulation stylet may include a reversible tissue anchor. In other examples, neither the catheter nor the bioelectric neuromodulation stylet may include a reversible tissue anchor. Dual-action catheter system 165 may be selectively coupled at a proximal end (e.g. end opposite the tip of the catheter) to a decoupler (not shown at FIGS. 1B-1C but see FIG. 2C). It may be understood that any number of combinations of tissue anchor(s) and decoupler (e.g. decoupler and no tissue anchor(s), decoupler with catheter anchor but without stylet anchor, decoupler with stylet anchor but without catheter anchor, decoupler with both catheter anchor and stylet anchor, catheter anchor alone without decoupler and without stylet anchor, stylet anchor alone without catheter anchor and without decoupler, no decoupler and no tissue anchors, stylet anchor and catheter anchor but no decoupler, etc.) are envisioned by the present disclosure. As mentioned above and which will be elaborated in further detail below, dual-action catheter system 165 may be used to provide bioelectric neuromodulation and/or pharmacological treatments to one or more patient(s) (e.g. 168), represented here as dashed line 160.

Control of a plurality of parameters and settings for dual-action catheter system 165 may be via controller 162. Controller 162 may further include instructions for providing the bioelectric neuromodulation and/or pharmacological treatment to the patient, which will be elaborated in further detail below. It may be understood that dual-action catheter system 165 may provide bioelectric neuromodulation and/or pharmacological treatment to the patient in a totally-automated fashion, partially-automated fashion, or in some examples via manual operation without relying on automation. Accordingly, a number of parameters related to pain management may in some examples be provided to controller 162 via patient-input to computing device 175 (e.g. desktop computer, smartphone, tablet, laptop, etc.), where computing device 175 stores an pain management application 166 (also referred to herein as “application”, or “software application”) for receiving the various parameters, and where controller 162 receives said parameters via wired or wireless network 176. The application may be communicably coupled to controller 162, such that via the application, control over bioelectric neuromodulation and/or pharmacological treatment may be enabled. As one example, such a parameter may comprise a current level of pain that the patient is experiencing. As another example, via the application, patient 168 may request an increased or decreased amount of bioelectric neuromodulation and/or pharmacological treatment (e.g. in an example where control over the dual-action catheter system is partially-automated or manually controlled).

Input to the application may in other examples additionally or alternatively be via administrator 178. Administrator 178 may comprise a physician, nurse, technician, etc. In some examples, administrator 178 may input information into the software application via computing device 175, in response to information received (e.g. orally, verbally, etc.) from patient 168, as represented by dashed line 180. However, in other examples, administrator 178 may input information into the software application via computing device 175, without receiving information from patient 168.

Application 166 may in some examples provide relevant information pertaining to pain management to patient 168 and/or administrator 178. For example, information pertaining to sensed electrical activity from the dual-action catheter system in response to or prior to providing one or more of bioelectric neuromodulation and/or pharmacological treatment may be communicated via controller 162 to software application 166, which may then be accessed by patient 168 and/or administrator 178.

Data, settings, parameters, patient information, etc., retrieved from controller 162 and/or input via application 166 may in some examples be stored on a remote and/or local server 190. In other examples, additionally or alternatively, such data, settings, patient information, etc., retrieved from controller 162 and/or input via application 166 may be stored on a cloud-based server 185. It may be understood that in some examples remote and/or local server 190 may comprise a same server as cloud-based server 185.

It may be understood that data stored at remote/local server 190 and/or cloud-based server 185 may comprise data (e.g. sensed neural activity patterns in response to particular pain management treatments), settings, parameters, patient information, etc., from one or more patients. Furthermore, in some examples, data stored on remote/local server 190 and/or cloud-based server 185 may include health-related data 177 from one or more patients (e.g. patient 168). Health-related data may comprise data pertaining to electronic health records (e.g. physician visit reports, lab results, etc.), and may further comprise data including but not limited to “omics” data (e.g. genomics, epigenomics, proteomics), imaging data (e.g. histology imaging, tissue imaging, blood smear imaging, etc.), scan data (e.g. magnetic resonance imaging (MRI) scans, positron emission tomography (PET) scans, computed tomography (CT) scans, etc.), ultrasound data, blood/plasma data, etc. Such health-related data may comprise information that may be useful in terms of predicting or inferring how a particular patient or patients may respond to particular bioelectric neuromodulation and/or pharmacological treatment, what level of pain that a particular patient or patients are expected to experience as a result, for example, of a surgical procedure (e.g. both during and after the procedure), likelihood that a particular patient or patients may develop a dependency, for example, for particular pharmacological treatments, expected efficacy of particular bioelectric neuromodulation strategies and/or particular pharmacological treatments, etc. In turn, for examples of the pain management system 100 where delivery of bioelectric neuromodulation and/or pharmacological treatment comprises total automation, or partial automation, any number of relevant parameters, settings, thresholds, etc., may be updated/modified as a function of such information inferred from the health-related data 177 and/or information retrieved from software application 166 and controller 162. In some examples, updates to such relevant parameters may require administrator approval, whereas in other examples such updates may be allowed without administrator approval.

More specifically, in order to predict or infer any number of the above-mentioned variables with regard to effective settings/parameters, pain level, dependency, etc., analytics module 195 may be used to access data stored on the remote/local server and/or cloud-based server, in order to process the data in such a way as to make the above-mentioned predictions. In one example, analytics module 195, through one or more of data mining methodology, machine learning methodology, deep learning methodology, etc., may learn that patients with a particular genetic mutation, for example, are likely to develop dependence for a particular pharmacological treatment, as opposed to another pharmacological treatment. As examples, machine learning methods and data analysis which may be used via analytics module 195 may include but are not limited to decision tree methods and linear regression, nonlinear regression, focusing projection, relevance, supported vector machines, Bayesian classifier, decision tree methods, logistic regression, neural networks, k-nearest neighbors, random forest, emergent self-organizing maps, artificial neural networks, etc.

As another representative example, analytics module may learn that patients of a certain age and particular genetic makeup are expected to respond well to bioelectric neuromodulation (e.g. certain learned frequencies, amplitudes, durations, etc.) while pharmacological treatments are less effective for a particular reported or inferred pain level. In such an example, settings, parameters, thresholds, etc., for such patients may be updated via the software application, and then the controller may control the providing of bioelectric neuromodulation and/or pharmacological treatments to the particular patients accordingly. In examples administrator 178 may further refine and/or confirm such settings, parameters, thresholds, etc.

As another example, software application may include one or more variable settings which a patient may control in order to manage pain, and may further include options for the patient to input a satisfaction level as to how effective a particular bioelectric neuromodulation and/or pharmacological treatment was. Such information may be correlated with sensed neural activity recorded via the bioelectric neuromodulation stylet, and taken together, analyzed via the analytics module 195 in order to predict based on recorded neural activity, how effective a particular treatment option may be.

While a few examples have been provided, a precise description of each and every potential scenario which may result from applying, for example, machine learning techniques via analytics module 195, is outside the scope of the present disclosure. However, it may be understood that based on such learning techniques conducted on data sets retrieved from software application 166, controller 162, and/or health-related data 177, control over the providing of bioelectric neuromodulation and/or pharmacological treatments may be updated/adjusted and applied to patient(s) accordingly. For example, thresholds related to frequency, duration, and amount of pharmacological treatment provided to patient(s) may be adjusted based on such learning. In another example, frequency and/or duration for providing bioelectric neuromodulation to patient(s) (and any number of parameters related to the providing of bioelectric neuromodulation) may be adjusted/updated in response to particular learned information. Said another way, based on such learned information, an optimal strategy for pain management for individual patients may be generated. The optimal strategy may comprise updating any number of settings/parameters, thresholds, etc., at software application 166, whereby controller 162 may retrieve such updated parameters/setting, and/or thresholds in order to provide bioelectric neuromodulation and/or pharmacological treatments in a manner expected to optimally manage pain for particular patient(s), while simultaneously avoiding issues related to tolerance, dependency, etc.

Turning to FIG. 1B, depicted is an example illustration 102 of a dual-action catheter system 165 of the present disclosure. Depicted is catheter 105, which may be used/implanted under a skin 110 of a patient, for a variety of reasons or medical procedures. Such a system may be implanted pre-operatively, intra-operatively, or post-operatively as desired. Specifically, a distal end 107 of catheter 105 is implanted under skin 110. Catheter 105 includes a sheath 112, and a hub 113 that includes male luer 114 connected at a proximal end 106 of catheter 105. In some examples (as will be discussed in further detail below at FIGS. 4A-4C), catheter 105 may include tissue anchor 115. A distal end 123 of bioelectric neuromodulation stylet 120 may be inserted into catheter 105, as depicted via arrow 121. Bioelectric neuromodulation stylet 120 may be connected at a proximal end 124 to a hub 125 with female luer 126 and male luer 127. When inserted into catheter 105, male luer 114 may engage or lock with female luer 126. Hub 125 may include electrical input source 130, which may connect via wired or wireless communication 132 to stimulating source 134. Stimulating source 134 may be under control of controller 162 via wired or wireless connection, as indicated via dashed line 136, and may be powered via a rechargeable battery or wall outlet. Electrical input source 130 may be electrically coupled to bioelectric neuromodulation stylet 120. While the discussion above relates to the use of male and female luers, it may be understood that in such examples and any other examples below, other connecting means (e.g. other connectors), for example NRFit connectors (B. Braun Medical Inc.), may be employed without departing from the scope of this disclosure.

In some examples, a tip end 137 of bioelectric neuromodulation stylet 120 may conduct electricity. It may be understood that tip end 137 may comprise a portion of bioelectric neuromodulation stylet 120 that is within a threshold distance from a tip 135 of bioelectric neuromodulation stylet 120, where tip 135 may be understood to be positioned at a most distal point of bioelectric neuromodulation stylet 120 in relation to hub 125. In some examples, the tip 135 may conduct electricity. In an example where tip 135, or tip end 137 conducts electricity, the remaining aspects of bioelectric neuromodulation stylet 120 can either conduct electricity, or may be electrically insulated or isolated. Electricity at tip 135 (or tip end 137) of bioelectric neuromodulation stylet 120 may comprise one of a single location or source, multi-location/multi-source. Electricity at tip 135 (or tip end 137) may be provided via one or more of a sequential, parallel and/or spiral arrangement. Furthermore, tip 135 (or tip end 137) may comprise a blunt end or, in other words, a domed tip, for easy insertion into a lumen 116 of catheter 105. Use of a blunt end may lower a risk of cutting/shearing of catheter 105 along lumen 116 during insertion of bioelectric neuromodulation stylet 120 into catheter 105, particularly when such insertion is conducted with catheter 105 already implanted.

Bioelectric neuromodulation stylet 120 may vary in shape, size and/or length, but it may be understood that a diameter of bioelectric neuromodulation stylet 120 may be smaller than a diameter of catheter 105. In this way, bioelectric neuromodulation stylet 120 may be readily inserted into catheter 105, as depicted via arrow 121.

In some examples, tip 135 (or tip end 137) of bioelectric neuromodulation stylet 120 may protrude distal end 107 of catheter 105. Said another way, bioelectric neuromodulation stylet 120 may be longer than catheter 105. In other examples, tip 135 (or tip end 137) of bioelectric neuromodulation stylet 120 may be flush with distal end 107 of catheter 105. Said another way, bioelectric neuromodulation stylet 120 may be of a substantially same length as catheter 105. As will be discussed in further detail below, bioelectric neuromodulation stylet 120 may in some examples include a deployable anchor positioned at or near tip 135 of the bioelectric neuromodulation stylet. For example, the deployable anchor may in some examples be within the portion of bioelectric neuromodulation stylet 120 comprising tip end 137. However, in other examples, the deployable anchor may be within a portion 155 of bioelectric neuromodulation stylet that protrudes past distal end 107 of catheter 105, where portion 155 is of a length longer than tip end 137.

Bioelectric neuromodulation stylet 120 may be comprised of metal, or metal in combination with another material or materials, depending on desired rigidity, flexibility and/or electrical conductivity. As one example, bioelectric neuromodulation stylet 120 may be at least as flexible as catheter 105, so as to not introduce greater rigidity (e.g. decreased flexibility, decreased mobility/flexibility in response to patient movement, “kicking” of the catheter in response to patient movement, etc.) in the catheter when the bioelectric neuromodulation stylet is inserted therethrough. Furthermore, as mentioned above, an important aspect of the stylet is that the distal end is blunt and not sharp, and that there are no aspects of the bioelectric neuromodulation stylet which may potentially compromise the catheter. In terms of electrical conductivity, desired characteristics may include the stylet being of a conductivity great enough to not have an undesirable voltage drop over the length of the stylet. Such an issue of an undesirable voltage drop may be routinely overcome by use of any metal deemed usable for catheter implants. However, there may be some examples where such an undesirable voltage drop may be relevant, such as in a circumstance where the bioelectric neuromodulation stylet includes a conductive polymer, or a polymer infused with a conductive material (e.g. carbon nanotubes). Thus, in such circumstances it may be understood that the design of the bioelectric neuromodulation stylet may account for avoiding an undesirable voltage drop over the length of the bioelectric neuromodulation stylet. In some examples, bioelectric neuromodulation stylet 120 may be hollow, whereby medication such as local anesthetics, cryoagents, etc., may be delivered therethrough. In other examples however, bioelectric neuromodulation stylet 120 may not be hollow and instead may be solid, such that medication may not be delivered through bioelectric neuromodulation stylet 120. As will be discussed in further detail below, in cases where bioelectric neuromodulation stylet 120 is solid such that medication cannot be delivered through bioelectric neuromodulation stylet 120, medication may be delivered via the distal end 107 of catheter 105 via a space between bioelectric neuromodulation stylet 120 and an inner aspect or inner wall of catheter 105.

Turning to FIG. 1C, an example illustration 150 is shown, depicting bioelectric neuromodulation stylet 120 inserted into catheter 105. In example illustration 150, tip 135 of bioelectric neuromodulation stylet 120 protrudes from distal end 107 of catheter 105. A portion 155 of bioelectric neuromodulation stylet that tip 135 protrudes distal end 107 of catheter 105 is a function of a length of bioelectric neuromodulation stylet 120 in relation to a length of catheter 105. Male luer 114 is engaged with female luer 126 of hub 125 when bioelectric neuromodulation stylet 120 is inserted into catheter 105 as depicted at FIG. 1C. In some examples, it may be understood that hub 125 may be adjustable along the length of bioelectric neuromodulation stylet 120, to accommodate catheters of varying lengths. In this way, an amount whereby the tip (e.g. 135) of the bioelectric neuromodulation stylet (e.g. 120) protrudes from the distal end (e.g. 107) of the catheter (e.g. 105) may be customized for each particular catheter.

Turning now to FIG. 1D, an example illustration 147 depicts a slightly more detailed view of aspects of pain management system 100. Depicted is dual-action catheter system 165. It may be understood that dual-action catheter system 165 includes bioelectric neuromodulation stylet 120, pharmacological delivery pathway 148, and catheter 116. In some examples, bioelectric neuromodulation stylet 120 may further include a deployable stylet anchor 141. However, in other examples bioelectric neuromodulation stylet 141 may not include stylet anchor 141, without departing from the scope of this disclosure. Accordingly, stylet anchor 141 is represented as a dashed box. In some examples, bioelectric neuromodulation stylet may further be selectively mechanically coupled to stylet decoupler 142, via a stylet decoupler connector 144.

Dual-action catheter system 165 may further include catheter 116. In some examples, catheter 116 may include a deployable catheter anchor 115. However, in other examples catheter 116 may not include catheter anchor 115, without departing from the scope of this disclosure. Accordingly, catheter anchor 115 is depicted as a dashed box. Furthermore, in some examples, catheter 115 may further be selectively mechanically coupled to catheter decoupler 143, via a catheter decoupler connector 145.

Dual-action catheter system may further include pharmacological delivery pathway 148, for delivering pharmacological treatments to a desired tissue site of a patient. Accordingly, pharmacological delivery pathway 148 may receive pharmacological treatments from pharmacological delivery pump 191. Dual-action catheter system 165 may thus include a pharmacological delivery connector 188 that functions to receive pharmacological treatments via pharmacological delivery pump 191 for delivery via pharmacological delivery pathway 148. Pharmacological delivery pump 191 may further include pump connector 189, for receiving input as to how to control operation of the pump from controller 162. As discussed herein, in examples where bioelectric neuromodulation stylet 141 is hollow, pharmacological delivery pathway may be through the hollow portion of the bioelectric neuromodulation stylet, for delivery via the tip (e.g. 135) of bioelectric neuromodulation stylet. In some examples where the bioelectric neuromodulation stylet is hollow and includes a stylet anchor, one or more additional delivery pathway(s) (e.g. see 456 at FIG. 4C) may be included. The one or more additional delivery pathway(s) may be understood to be included within a vicinity of stylet anchor 141, such that the one or more additional delivery pathway(s) become exposed to a desired tissue site for delivery of pharmacological treatments when the stylet anchor is deployed, but which are not exposed to the desired tissue site when the stylet anchor is not deployed.

In examples where the bioelectric neuromodulation stylet is solid, it may be understood that pharmacological delivery pathway 148 is via a space defined as between stylet 120 and inner walls of catheter 116.

Bioelectric neuromodulation stylet 120 may include electrical input source 130, which may operate to receive instructions from controller 162 for controlling delivery of bioelectric neuromodulation to a patient. Bioelectric neuromodulation stylet may further include electrical output source 187, whereby sensed neural activity from tissue via electrodes of the bioelectric neuromodulation stylet may be communicated to controller 162.

Information including but not limited to sensed electrical activity, parameters for delivery bioelectric neuromodulation and pharmacological treatments, personalized thresholds, personalized settings, etc., may be output from controller 162 to pain management application 166, as depicted via arrow 184. Controller may further receive such information from pain management application 166 as depicted via arrow 183. In some examples, data retrieved from controller 162 and/or pain management application 166 may be sent to analytics module 195, as depicted via arrows 185 and 181, respectively. Via a machine learning algorithm operating on said data, analytics module 195 may output learned information (e.g. information pertaining to adjusted personalized thresholds) to pain management application 166 and/or controller 162, as depicted via arrows 182 and 186, respectively. It may be understood that personalized thresholds which may be adjusted or refined based on output from analytics module 195 may include but are not limited to frequency of delivery of bioelectric neuromodulation, frequency of delivery of pharmacological treatments, time duration between particular treatments, duration of bioelectric neuromodulation treatments, duration of pharmacological treatments, specific parameters related to delivery of bioelectric neuromodulation (e.g. pulse frequency, current amplitude), concentration of pharmacological treatment delivered to a patient, type of pharmacological treatment (e.g. drug type), etc.

As discussed above, bioelectric neuromodulation stylet 120 may be capable of delivering electricity to tissue, and there may be additional means for delivering medication to said tissue while bioelectric neuromodulation stylet 120 is inserted into catheter 105. Accordingly, turning to FIG. 2A, it depicts an example illustration 200 where bioelectric neuromodulation stylet 120 is inserted into catheter 105, as discussed above with regard to FIG. 1B. Tip 135 protrudes from distal end 107 of catheter 105. It may be understood that stylet 120 is hollow in example illustration 200. Accordingly, medication may be delivered to tissue through bioelectric neuromodulation stylet 120, as indicated via arrow 205. Medication may be provided to bioelectric neuromodulation stylet 120 via syringe 210. Specifically, a female luer 215 corresponding to syringe 210 may couple to male luer 127 associated with bioelectric neuromodulation stylet 120, where syringe 210 may be loaded with medication and then delivered to tissue via bioelectric neuromodulation stylet 120. Bioelectric neuromodulation stylet 120 may further deliver electrical pulses to tissue via electrical energy provided to bioelectric neuromodulation stylet 120 via stimulating source 134. While a syringe is depicted at FIG. 2A, it may be understood that in other examples a tubing (not shown) may couple to male luer 127, where medication may be provided via a pump (not shown) that pumps medication from a reservoir (not shown) capable of storing the medication. It may be understood that such a pump may be under control of a controller (e.g. 162), where the controller receives instructions as to how to deliver pharmacological treatments based on information provided to the controller via the software application.

In other examples bioelectric neuromodulation stylet 120 may not be hollow. Turning to FIG. 2B, it depicts an example illustration 250, where bioelectric neuromodulation stylet 120 is inserted into catheter 105. Tip 135 protrudes from distal end 107 of catheter 105. In this example illustration, bioelectric neuromodulation stylet 120 is solid, and thus instead of medication being delivered to tissue through bioelectric neuromodulation stylet 120, medication is instead delivered to tissue via a space or spaces between bioelectric neuromodulation stylet 120 and sheath 112, as exemplified via arrows 255. Said another way, medication may be delivered to tissue via lumen 116 defined by bioelectric neuromodulation stylet 120 and sheath 112.

While not explicitly illustrated at FIGS. 1B-2B, it may be understood that the dual-action catheter systems of the present disclosure may in some examples include an option to mechanically couple such systems to a decoupler in order to isolate implanted portions of the cathether and/or bioelectric neuromodulation stylet from undergoing undesirable movement. Turning to FIG. 2C, it depicts an example illustration 260 of a decoupler 261. While not explicitly illustrated at FIGS. 1B-2B, it may be understood that such a decoupler may be mechanically coupled to one of the catheter hub (e.g. 113) or stylet hub (e.g. 125), via suitable connecting means (e.g. NRFit connectors, luer-style connectors, etc.). Accordingly, connector 262 is depicted at FIG. 2C. It may be understood that the decoupler, or decoupling system may be positioned between a proximal end of the catheter/bioelectric neuromodulation stylet (the proximal end opposite of a distal end 107) and utility tubing 263, and may be coupled or attached temporarily to a patient's skin at an exit site of the catheter. Via the use of such a decoupler, a risk of stressing any sites of the catheter/bioelectric neuromodulation stylet that are anchored, may be reduced/minimized. Such a decoupling mechanism may in some examples allow for absorbing of external forces and impacts, such as changing of utility tubing, accidental pulling of utility tubing, etc. The decoupler may be designed to have varying amounts of motion. For example, a range of distance/movement that the decoupler allows may differ depending on application, location on body, purpose, etc. A predetermined allowed range of motion allowable by the decoupler may range from zero to a maximum anticipated distance desired for each particular application.

As mentioned above and which will be further elaborated below, in line with the description herein, the catheter may include an anchor or tissue lock, and the bioelectric neuromodulation stylet may additionally or alternatively include an anchor or tissue lock. Further, as discussed, the proximal end of catheter systems of the present disclosure may include an option for mechanically coupling to a decoupler or in other words, to a decoupler system. Thus, it is herein recognized that there may be a variety of options for combining tissue lock(s) and/or decouplers consistent with the present disclosure. Accordingly, turning to FIG. 2D, a chart 280 depicts example combinations of decoupler, catheter anchor and bioelectric neuromodulation stylet anchor of the present disclosure. Specifically, example A depicts use of a decoupler with the dual-action catheter system of the present disclosure, in the absence of catheter anchor and stylet anchor. Example B depicts use of a decoupler and a catheter anchor, where the stylet does not include an anchor. Example C depicts use of a decoupler and a stylet anchor, where the catheter does not itself include an anchor. Example D depicts a situation where the dual-action catheter system relies on a decoupler, catheter anchor and stylet anchor. Example E depicts use of a catheter anchor, but where a decoupler is not used and where the stylet is not anchored. Example F depicts a situation where both the stylet is anchored as well as the catheter. Example G depicts an example where only the stylet is anchored, but not the catheter and where a decoupler is not relied upon. Finally, in some examples the dual-action catheter systems of the present disclosure may be used in the absence of decoupler, catheter anchor, and stylet anchor, exemplified by Example H.

It may be understood that where use of a decoupler is indicated, the decoupler may be mechanically coupled to either the bioelectric neuromodulation stylet, or the catheter itself. More specifically, Example B depicts use of the decoupler with the catheter anchor. In such an example, it may be understood that the decoupler may be mechanically coupled to either the catheter, or to the bioelectric neuromodulation stylet. Similar reasoning applies to Example C. In a case where the decoupler is utilized under situations where both the catheter is anchored and the stylet is anchored (e.g. Example D), either the catheter may be mechanically coupled to the decoupler, the stylet may be mechanically coupled to the decoupler, or both may be coupled to individual decouplers.

It may be understood that the combinations with regard to FIG. 2D may relate to use of the dual-action catheter, rather than to physically whether or not the stylet or catheter includes an anchor. For example, in a situation where the decoupler is relied upon but where the catheter is not anchored and where the stylet is not anchored (e.g. Example A at FIG. 2D), it may be understood that in one example, the catheter and/or the stylet may include an anchoring means that is simply not deployed. In other examples, neither the catheter nor the stylet may have any means for anchoring the catheter and/or stylet.

Turning now to FIG. 3A, example illustration 300 depicts in further detail bioelectric neuromodulation stylet 120. As discussed above, bioelectric neuromodulation stylet 120 may couple to hub 125, where hub 125 includes female luer 126, and may additionally include male luer 127 (not shown at FIG. 3A but see FIG. 3B). Inset 310 depicts a close-up view of tip 135 of bioelectric neuromodulation stylet 120. In this example illustration, a single core flexible wire 315 is molded into a flexible extrusion (e.g. plastic). Thus, in this example, stylet 120 is solid. A side view of stylet 120 is depicted at FIG. 3B, where male luer 127 is shown coupled to hub 125. It may be understood that male luer 127 may couple to a means (e.g. syringe) for delivering medication to tissue when bioelectric neuromodulation stylet 120 is inserted into a lumen (e.g. 116) of a catheter (e.g. 105).

Turning now to FIGS. 4A-4C, example embodiments of select aspects of a dual-action catheter system (e.g. 165) of the present disclosure are depicted. FIG. 4A depicts an example illustration 400, depicting catheter 405. It may be understood that catheter 405 as depicted at FIG. 4A may comprise the same catheter as catheter 105 depicted at FIG. 1B. Catheter 405 comprises outer sheath 410 surrounding inner sheath 411. Outer sheath 410 comprises a plurality of lengthwise slits (not numbered) that define a plurality of malecot extensions 412. Malecot extensions 412 comprise living hinges 413. Thus, FIG. 4A depicts a dual-sheath catheter. Inner sheath 411 defines a lumen (e.g. 116) whereas outer sheath 410 comprises a tissue lock 414 or anchor. The lumen may be capable to receive (e.g. arrow 121) bioelectric neuromodulation stylet 120. For simplicity, only bioelectric neuromodulation stylet 120 and female luer 126 are depicted, however it may be understood that bioelectric neuromodulation stylet 120 may include other components as discussed above with regard to FIGS. 1B-1C. In such examples, fluid (e.g. pharmacological treatment) may be delivered either via the bioelectric neuromodulation stylet itself as depicted at FIG. 2A, or via spaces between the bioelectric neuromodulation stylet and the inner sheath (similar to that depicted at FIG. 2B).

FIG. 4A depicts tissue lock 414 in an extended position. Tissue lock 414 may also adopt a collapsed position (not shown at FIG. 4A). Said another way, outer sheath 410 comprises a tissue lock 414 movable between a collapsed position and an extended position (shown at FIG. 4A), where the tissue lock 414 forms a reversible tissue anchor when in the extended position. Where catheter 405 comprises a same catheter as catheter 105, it may be understood that tissue lock 414 may comprise a same tissue lock as anchor 115. An actuator (not shown at FIG. 4A) connected to proximate end 415 of catheter 405 may be configured to activate tissue lock 414 by sliding outer sheath 410 length-wise relative to inner sheath 411. While not explicitly illustrated, it may be understood that in some examples a coil may be embedded within inner sheath 411. Such a coil may in some examples be configured to do one or more of the following. The coil may be configured to 1) provide echogenicity, 2) provide a bidirectional antenna configured to deliver electrical energy to nervous tissue and to transmit internal electricity from nerve electrical activities, and/or 3) improve anti-kinking of catheter 405 in locations prone to kinking. Such a coil may thus in some examples have varied wraps per inch length-wise or varied thickness along a length of the coil. However, such a coil may not be included for catheter 405 without departing from the scope of this disclosure.

As discussed above, bioelectric neuromodulation stylet 120 may in some examples be hollow, or in other words the stylet itself may include a lumen (e.g. fluid delivery lumen), or may be solid. Furthermore, as discussed above, bioelectric neuromodulation stylet 120 may in some examples itself include a tissue anchor (for cases where bioelectric neuromodulation style 120 extends past a tip or distal end (e.g. 107) of the catheter. Accordingly, turning to FIG. 4B, it depicts an example embodiment 450 of bioelectric neuromodulation stylet 120. In example embodiment 450, stylet 120 includes an inner sheath 426 and an outer sheath 427. In other words, in this example, bioelectric neuromodulation stylet 120 comprises a similar design as that of catheter 405 discussed above at FIG. 4A. In this way, a relative movement of inner sheath 426 with respect to outer sheath 427 may result in stylet tissue anchor 485 (e.g. same as 141) being deployed. Furthermore, in example embodiment 450 of bioelectric neuromodulation stylet 120, inner sheath 426 includes one or more holes or passageways 456. When stylet tissue anchor 485 is in a collapsed state, a fluid delivery lumen 457 in the vicinity of tissue anchor 485 may not be exposed to tissue (under conditions when the stylet is inserted through a lumen (e.g. 116) of a catheter (e.g. 105). However, upon deployment of tissue anchor 485 (depicted at FIG. 4B), fluid delivery lumen 457 may become exposed to tissue in the vicinity of the tissue anchor. Accordingly, when tissue anchor 485 is in a collapsed state, fluid (e.g. pharmacological treatment) may be delivered through fluid delivery lumen 457 and may exit the bioelectric neuromodulation stylet at the tip 135, and may not exit through the one or more holes or passageways 456. Alternatively, when tissue anchor 485 is deployed (depicted at FIG. 4B), fluid may be delivered through fluid delivery lumen 457 and may exit the bioelectric neuromodulation stylet at tip 135 and in the vicinity of anchor 485, via the one or more holes or passageways 456. In this way, under circumstances where bioelectric neuromodulation stylet 120 is hollow, extends past a tip (e.g. distal end 107) of a catheter (e.g. 105), and includes a deployable tissue anchor of the form depicted at FIG. 4B, fluid delivery to tissue may be via selectable routes depending on whether the tissue anchor is deployed or not.

Further depicted at FIG. 4B are electrode bands 476. Specifically, as discussed above with regard to FIG. 1B, tip 135 of bioelectric neuromodulation stylet 120 may conduct electricity. Accordingly, electrode bands 476 are depicted at tip 135. In this example, the remaining aspects of the bioelectric neuromodulation stylet are electrically isolated, however in other examples remaining aspects of the stylet may too conduct electricity. A length 451 between tip 135 and tissue anchor 485 may be variable depending on the design and desired use.

Turning now to FIG. 4C, in another example embodiment 480 of bioelectric neuromodulation stylet 120, the stylet is depicted as not being hollow, in other words the stylet is solid, exemplified by diagonal lines 481. In such an example, whether tip 135 is blunt with a tip of a catheter (e.g. 105) or extends past the tip of the catheter, it may be understood that fluid delivery to tissue when the stylet is inserted through a lumen of the catheter may be via space in the lumen of the catheter that is not occupied via the stylet. In other words, fluid delivery (e.g. pharmacological treatment) may be around the stylet through the lumen of the catheter, and not through the stylet itself, as discussed above with regard to FIG. 2B. In other words, when the stylet is solid, whether or not the anchor 485 is deployed, there is no means for fluid delivery at the site of the anchor as compared to the tip, as fluid delivery is simply around the outside of the stylet through the lumen of the catheter through which the stylet is inserted.

FIG. 4C further depicts outer sheath 427 and inner sheath 426, similar to that discussed for FIG. 4B. Thus, tissue anchor 485 of bioelectric neuromodulation stylet 485 may be deployed/collapsed as discussed above with regard to FIG. 4B. A length 451 between tip 135 of the stylet and the anchor may be variable, depending on the application. One or more electrodes 476 may be positioned at tip 135.

While the FIGS. 4B-4C depict examples where the bioelectric neuromodulation stylet includes tissue anchor 485, in other examples such a stylet may not include means for deploying such a tissue anchor without departing from the scope of this disclosure. Furthermore, while depicted for each of catheter 405 depicted at FIG. 4A, stylet 120 depicted at FIG. 4B, and stylet 120 depicted at FIG. 4C is a deployable anchor deployable via relative movement of an inner sheath with respect to an outer sheath, it may be understood that such designs are not meant to be limiting and other designs for each of the catheter and the bioelectric neuromodulation stylet are encompassed by the present disclosure. For example, the catheter may include an inflatable balloon anchor, barb, hook, protrusion, etc., rather than the type of anchor (e.g. malecot anchor) depicted at FIG. 4A. Similarly, the bioelectric neuromodulation stylet may include an inflatable balloon anchor, barb, hook, protrusion, etc., rather than the type of anchor (e.g. malecot anchor) depicted at FIGS. 4B-4C.

Regardless of the specific design of the bioelectric neuromodulation stylet (e.g. solid, hollow, with anchor, without anchor), electrical capability provided at tip 135 may in some examples comprise 360-degree electrode(s) for conducting electricity. The electrode(s) at the tip may be capable of accommodating both evoked motor and evoked sensorial stimulation. For example, for evoked motor stimulation the electrode(s) may accommodate up to 5 mA (0.2-5 mA). As another example, for sensorial stimulation, the electrode(s) may accommodate various frequencies including but not limited to ≤1K Hz, ≤3K Hz, ≤5K Hz, ≤10K Hz, ≤20K Hz, ≤50K Hz, ≤100K Hz etc. Sensorial stimulation may comprise variable energy levels, for example 0.05 mA up to 5 mA, depending on clinical need. Said another way, the dual-action catheter system of the present disclosure may provide stimulation in a range of 0-100K Hz, with variable energy levels comprising 0.05 mA up to 5 mA. More specifically, in line with the pain management systems discussed herein, for frequencies ≤10K Hz, energy levels may be in one of the following ranges 0.05 mA up to 5 mA, 0.05 mA up to 1 mA, 0.05 mA up to 0.5 mA, 0.05 up to 0.1 mA. For frequencies ≤5K Hz, energy levels may be in one of the following ranges 0.05 mA up to 5 mA, 0.05 mA up to 1 mA, 0.05 mA up to 0.5 mA, 0.05 up to 0.1 mA. For frequencies ≤3K Hz, energy levels may be in one of the following ranges 0.05 mA up to 5 mA, 0.05 mA up to 1 mA, 0.05 mA up to 0.5 mA, 0.05 up to 0.1 mA. For frequencies ≤1K Hz, energy levels may be in one of the following ranges 0.05 mA up to 5 mA, 0.05 mA up to 1 mA, 0.05 mA up to 0.5 mA, 0.05 up to 0.1 mA.

The stimulating source (e.g. 134) for the catheter systems discussed herein may include one or more of a number of characteristics. For example, the stimulating source may in some examples comprise a battery-operated stimulation source, capable to generate electric pulse(s) at predetermined frequency, intensity and/or duration. With regard to frequency, it may be grossly classified into several categories. Specifically, in one example frequency may be below a physiological nerve firing frequency. In another example, frequency may be at a physiological nerve firing frequency. In another example, frequency may be above a physiological nerve firing frequency. A typical stimulation frequency may comprise 20 Hz, for example, whereas higher frequency in a range of 1K to 50K Hz may be used for their differential nerve effect (sensorial, motor, sympathetic, etc.). Block threshold may comprise a linear function of the frequency over a range of 5-30K Hz. Frequency may comprise current-controlled frequency, whereas frequency may comprise voltage-controlled frequency in other examples without departing from the scope of this disclosure.

The stimulating source (e.g. 134) may comprise wired or wireless battery charging, and the battery powering the stimulating source may be disposable. In some examples, parameters (e.g. frequency, intensity and/or duration) for the stimulating source may be received at the stimulating source via wired or wireless communication. Control over such parameters may in some examples be via a computing device including but not limited to a smart phone, laptop, tablet, etc. In some examples, remote management of the stimulating source may be enabled via wireless technology (e.g. Bluetooth).

The catheter systems of the present disclosure are suitable for indwelling nerve block applications. In some examples, a catheter of the present disclosure may be extravascularly implanted an appropriate distance from a target nerve. For example, an appropriate distance (measuring catheter tip to nerve) may be less than or equal to 1, 0.8, or 0.5 mm. Methods for use, as discussed in further detail below, may include navigating a needle/catheter tip to within the appropriate distance, and then deploying a tissue lock (e.g. 115, 414), where relevant. Navigating the needle/catheter tip may include the presence of a needle (for example either within the catheter or housing the catheter), whereupon after the catheter is placed and the tissue lock engaged (e.g. actuated to an extended position), where relevant, the needle may be removed. In some examples the needle may comprise a stimulating needle whereas in other examples the needle may comprise a non-stimulating needle.

Accordingly, turning to FIG. 5, an example illustration 500 is depicted, illustrating an example needle 505 disposed within an example catheter 510. It may be understood that catheter 510 may be the same as catheter 105, or may be the same as catheter 405, without departing from the scope of this disclosure. Catheter 510 comprises lip 515, which may provide a block whereby needle 505 may push against. During placement of the catheter, as the catheter is inserted into a patient, the catheter may encounter an axial force in the direction of the solid black arrow 520. Lip 515 thus may prevent the catheter 510 from sliding relative to needle 505 during insertion. In examples where catheter 510 includes a tissue lock (e.g. 115, 414), by preventing sliding of catheter 510 relative to needle 505, premature deployment of the tissue lock may be avoided.

Turning now to FIG. 6, an example methodology 600 is shown, depicting steps for use of a dual-action catheter system comprising a bioelectric neuromodulation stylet, as disclosed above with regard to FIGS. 1B-4C. A first set of instructions 601 may comprise steps involved prior to a surgical operation on a patient, and a second set of instructions 602 may comprise steps involved post the surgical operation. However, while method 600 depicts pre- and post-operative steps, it may be understood that in other examples, a catheter may be implanted only after a surgical operation and not prior to the surgical operation, without departing from the scope of this disclosure.

Method 600 begins at 605, and includes a physician inserting a catheter and needle combo (see FIG. 5) extravascularly into the patient, and locating a desired nerve. The desired nerve may be located using one or more of ultrasound, nerve stimulation, or both. Once located, method 600 may proceed to 610, where the tissue lock (e.g. 115) may be deployed, under circumstances where the catheter includes a tissue lock and/or where such deployment is desired. As such, a step may be optional, step 610 is depicted as a dashed box.

Proceeding to 615, method 600 includes infusing a desired amount of a selected anesthetic to the site of the desired nerve. In one example, the anesthetic may be delivered via the needle (e.g. 505). Once the anesthetic has been delivered, method 600 may proceed to 620, where the needle may be removed from the catheter. However, in other examples the needle may be removed and then the anesthetic may be delivered via the lumen of the catheter, without departing from the scope of this disclosure. With the needle removed, the catheter may be optionally secured to a decoupler, depending on the particular procedure, at step 625. As step 625 is optional, it is depicted as a dashed box. Continuing to 630, method 600 may include securing the catheter and/or the decoupler to the patient's skin.

Between steps 630 and 635, it may be understood that the particular surgical procedure is conducted on the patient. Subsequently, at step 635, method 600 may include removing skin securement dressing that was used at step 630 to secure the catheter and/or decoupler to the patient's skin. If the decoupler was secured to the catheter at step 625, then at step 640, method 600 may include disconnecting the decoupler. However, as this step may not occur, step 640 is depicted as a dashed line, similar to that of step 625 which is also optionally performed.

Proceeding to 645, method 600 includes inserting the bioelectric neuromodulation stylet (e.g. 120) into the lumen (e.g. 116) of the catheter (e.g. 105). Once inserted, method 600 may proceed to 650, where the decoupler is connected to the neuromodulation device. Again, in some cases the decoupler may not be coupled to the bioelectric neuromodulation stylet, without departing from the scope of the present disclosure. Furthermore, while not explicitly illustrated, it may be understood that in some examples, at 645, following insertion of the bioelectric neuromodulation stylet, a tissue anchor associated with the stylet itself may be deployed.

At 655, method 600 may include optionally securing the catheter and inserted bioelectric neuromodulation stylet to the patient's skin. Once secured (or not in some examples), method 600 may proceed to 660, where the bioelectric neuromodulation stylet is connected to a nerve stimulation unit (e.g. stimulating source 134).

In this way, the catheter may be first used in a manner whereby the catheter is implanted in a patient and used pre-surgical operation in order to deliver anesthetics, and then after the surgical operation, the bioelectric neuromodulation stylet may be inserted into the catheter to be utilized for pain management. As discussed above with regard to FIGS. 2A-2B, medicine (e.g. pharmacological treatments) may be additionally or alternatively be delivered via the catheter while the neuromodulation device is inserted into the catheter.

Turning now to FIG. 7, an example method 700 is depicted, detailing methodology for patient pain management, where such pain management is at least partially automated, as discussed above. At least parts of method 700 may be carried out by a controller (e.g. 162), where the controller may store instructions for carrying out parts of method 700 in non-transitory memory. Instructions for carrying out at least parts of method 700 may be executed by the controller based on instructions stored on a memory of the controller and in conjunction with signals and/or instructions received from a software application (e.g. 166) and/or sensor(s) (e.g. sensing component of the bioelectric neuromodulation stylet). The controller may control a providing of pain management via one or more of bioelectric neuromodulation and/or pharmacological treatments, according to the method below.

Method 700 begins at 705, and may include, via instructions stored at the controller and in conjunction with one or more settings, parameters, etc., associated with the software application that is communicatively coupled to the controller, providing bioelectric neuromodulation and/or pharmacological treatments to a patient via the dual-action catheter system of the present disclosure. The providing of bioelectric neuromodulation and/or pharmacological treatments may be according to a schedule that is at least partially automated, however it may be understood that in other examples the schedule may be fully automated, in some examples with an option for a patient and/or administrator input. As discussed in detail above with regard to FIG. 1A, one or more parameters, settings, thresholds, etc., may be updated at the software application and communicated as instructions to the controller as a function of learning conducted via an analytics module (e.g. 195). As representative examples, learning via the analytics module may enable a prediction of a particular patient's pain level, prediction of how a particular patient will respond to particular bioelectric neuromodulation and/or pharmacological treatments, prediction of potential tolerance and/or dependency issues related to the providing of bioelectric neuromodulation and/or pharmacological treatments, etc. In turn, such predictions may enable at least partial automation of the setting of one or more thresholds associated with the providing of pain management to the patient, and at least partially automated control over settings and/or parameters for providing bioelectric neuromodulation and/or pharmacological treatments via the dual-action catheter system. As discussed above with regard to FIG. 1A, such predictions may be based on machine learning, for example, that relies on one or more data sources including but not limited to health-related data (e.g. 177), data input into the software application by the patient and/or administrator (e.g. particular parameters, settings, pain level(s), thresholds, satisfaction associated with particular pain management strategies, etc.), and/or neural activity data sensed via the bioelectric neuromodulation stylet as a function of provided bioelectric neuromodulation and/or pharmacological treatments. Furthermore, the providing of bioelectric neuromodulation and/or pharmacological treatments at 705 may be a function of input to the software application of certain parameters and/or settings, pain level, satisfaction associated with particular pain management strategies, etc., that are not dependent on or are in addition to the learned information. For example, such input may be via the administrator (e.g. 178) and/or patient. In some examples, the administrator may set and/or adjust thresholds at the software application, the thresholds associated with the providing of bioelectric neuromodulation and/or pharmacological treatments to the patient. As one example, a learned threshold or thresholds may be adjusted manually by the administrator in some examples. Similarly, learned parameters, settings, etc., may be in some examples adjusted manually by the administrator.

For the providing of bioelectric neuromodulation and/or pharmacological treatments at 705, it may be understood that in some examples, bioelectric neuromodulation may not be provided at the same time as pharmacological treatments. However, in other examples, bioelectric neuromodulation may be provided at the same time as pharmacological treatments. In some examples, providing of bioelectric neuromodulation may alternate with providing of pharmacological treatments, however in other examples one bioelectric neuromodulation treatment may follow another bioelectric neuromodulation treatment and/or one pharmacological treatment may follow another pharmacological treatment. In some examples, there may be a threshold duration set such that a particular treatment may not commence within the threshold duration of time since a prior treatment. For example, following a bioelectric neuromodulation treatment, a threshold duration may have to elapse in order to then provide another bioelectric neuromodulation treatment, or a pharmacological treatment. Such threshold durations may be variable. For example, the threshold duration between providing sequential bioelectric neuromodulation treatments may differ from a threshold duration between providing sequential pharmacological treatments. However, in other examples the threshold duration may be the same. In other examples, the threshold duration between following a pharmacological treatment with a bioelectric neuromodulation treatment, or vice versa, may be either the same or different than a threshold duration between sequential bioelectric neuromodulation treatment, or sequential pharmacological treatment.

Said another way, the threshold duration between providing pain management treatments (e.g. bioelectric neuromodulation and/or pharmacological treatments) may be variable. The variable nature of such threshold durations may be in some examples based on information learned via the analytics module, set and/or adjusted by the administrator, etc. Turning to FIG. 8, a timeline 800 is depicted, illustrating an example sequence of bioelectric neuromodulation treatments followed by a sequence of pharmacological treatments, in order to illustrate variable threshold durations between said treatments.

Accordingly, at time t0, the controller administers bioelectric neuromodulation to a patient. Such treatment proceeds until time t1, when such treatment is stopped. Between time t1 and t2, no treatments are provided, based on threshold duration 805. In other words, bioelectric neuromodulation and/or pharmacological treatments are prevented from being administered between time t1 and t2.

At time t2, the threshold duration 805 elapses, and bioelectric neuromodulation is once again provided. At time t3, said bioelectric neuromodulation is again stopped. No treatments are provided between time t3 and t4, as a function of the threshold duration 805. At time t4, bioelectric neuromodulation is once again provided after threshold duration 805 elapses.

At time t5, bioelectric neuromodulation is again stopped. A second threshold duration 810 prevents pharmacological treatment from being administered until the second threshold duration elapses. Said another way, the next scheduled treatment is pharmacological treatment, and thus, such treatment, when following bioelectric neuromodulation, may be administered after the second threshold duration 810 elapses. Accordingly, at time t6, when the second threshold duration 810 elapses, pharmacological treatment is provided. Such treatment is stopped at time t7. The next scheduled treatment comprises pharmacological treatment, however a third threshold duration 815 prevents pharmacological treatment from being administered following pharmacological treatment, until the third threshold duration 815 elapses.

Thus, as depicted at timeline 800, the first threshold duration 805 determines how long after bioelectric neuromodulation another treatment comprising bioelectric neuromodulation may be provided to the patient. The second threshold duration 810 determines how long after bioelectric neuromodulation a pharmacological treatment may be provided to the patient. The third threshold duration 815 determines how long after pharmacological treatment another pharmacological treatment may be provided to the patient. As depicted for timeline 800, the first threshold duration is different than the second threshold duration, which in turn is different than the third threshold duration. However, such an example is meant to be illustrative, and in other examples the threshold durations (e.g. 805, 810, 815) may be the same, two out of the three threshold durations may be the same, etc., without departing from the scope of this disclosure. Also, not depicted is a fourth threshold duration, which may determine how long after a pharmacological treatment a bioelectric neuromodulation treatment may be provided. The fourth threshold duration may be the same or different than any of the first through third threshold durations.

In some examples, the variable nature of the threshold durations may be adjusted based on one or more of information learned via the analytics module, in response to patient and/or administrator input into the software application (e.g. 166), as a function of neural activity data retrieved from the patient via the bioelectric neuromodulation stylet in response to particular treatments (e.g. bioelectric neuromodulation and/or pharmacological treatment), etc. For example, while a particular pain management schedule is being provided to the patient, threshold durations between particular treatments may be adjusted automatically, partially automatically, or manually (e.g. via the administrator).

Returning to FIG. 7, while bioelectric neuromodulation and/or pharmacological treatments are being provided to the patient in order to manage pain, method 700 may proceed to 710. At 710, method 700 may include the patient or administrator determining whether it is desired to change the way that the pain management is being conducted. If not, then method 700 may proceed to 715, where the current pain management schedule is maintained. Continuing to 718, method 700 may include collecting data pertaining to the current pain management schedule. Collecting data may include collecting and storing one or more parameters, settings, thresholds, etc., related to providing the pain management, at the servers (e.g. 185, 190). Collecting data may in some examples include retrieving data from the controller corresponding to neural activity (e.g. firing patterns) sensed by the bioelectric neuromodulation stylet, as a function of pharmacological and/or bioelectric neuromodulation treatments. Such data may be correlated in some examples with a current level of pain experienced by the patient, as input to the software application periodically by the patient and/or administrator. In some examples, the software application may periodically request input from the patient and/or administrator as to the current level of pain (e.g. pain of a level 1-10, 10 being highest and 1 being lowest) being experienced by the patient, for correlating sensed neural activity and provided bioelectric neuromodulation and/or pharmacological treatments with current pain level. By collecting such data, machine learning via the analytics module (e.g. 195) may enable a learning of optimal strategies for managing pain that is patient specific.

Returning to 710, in a case where a change to the pain management is desired, method 700 may proceed to 720. At 720, method 700 may include requesting a change to the pain management schedule via the software application. In some examples, such a request may be input to the software application via the patient. In other examples, such a request may be input to the software application via the administrator. As a representative example, the patient may be experiencing a level of pain for which the current pain management is not adequately addressing. For example the patient may be experiencing a higher level of pain that is not being mitigated by the current schedule. In another example the patient may have a low level of pain and may thus desire less in the way of provided bioelectric neuromodulation and/or pharmacological treatment. In some examples, the patient may communicate such information to the administrator, who may then input the conveyed information to the software application to request the change in pain management.

While the patient and/or administrator may request the change at 720, in other examples such a request may be in response to sensed neural activity via the bioelectric neuromodulation stylet. For example, based on information learned over time via the analytics module (e.g. 195), in response to a particular sensed neural activity it may be inferred that a level of pain that the patient is experiencing is not being adequately addressed. Said another way, the analytics module may allow for particular neural activity firing patterns to be associated with particular pain levels, such that in response to detection of such patterns, particular pain levels may be inferred and accordingly, if such pain levels are further inferred as not being adequately addressed via the current pain management schedule, then a request to change the schedule may be input to the software application automatically at 720 via communication between the software application and the analytics module.

With such a request received at the software application at 720, method 700 may proceed to 725. At 725, method 700 may include indicating as to whether the request exceeds certain predetermined thresholds and/or preset parameters. The thresholds may include, but are not limited to frequency in which a pharmacological treatment is provided, duration for which a particular pharmacological treatment is provided, amount/concentration of a particular pharmacological treatment, duration between particular treatments (e.g. pharmacological and/or bioelectric neuromodulation), frequency in which bioelectric neuromodulation is provided, parameters related to the providing of bioelectric neuromodulation (e.g. stimulation frequency, amplitude, applied current and duration, etc.), etc. Thresholds related to the above-mentioned variables may be set at the software application via the administrator, may be automatically set based on learned information via the analytics module, or may be set based on some combination of administrator input as a function of one or more of health-related patient data and/or learned data via the analytics module.

As one representative example, a patient may input a particular pain level that they are experiencing into the software application. In this example, the particular level of pain is of a level greater than that which the current pain management strategy is addressing. However, based on one or more of information learned via the analytics module, information input to the software application via the patient and/or administrator, etc., the software application determines that the request involves providing a level of pharmacological treatment (e.g. amount of particular pharmacological treatment, frequency with which the pharmacological treatment is provided, etc.) that exceeds one or more preset thresholds. For example, the patient may be someone prone to developing a dependency for a particular pharmacological treatment, and thus it may not be desirable to allow said patient to receive the particular pharmacological treatment at the level or frequency which would address the pain the patient has input to the software application.

Thus, at 725, in response to the request to change the pain management schedule being received at the software application, and further in response to the request exceeding one or more preset thresholds, method 700 may proceed to 730. At 730, method 700 may include sending the request to the administrator for approval. In other words, rather than the software application commanding the controller to change the pain management schedule in order to address the request, an alert may be sent (e.g. via text, email, audibly, etc.) to the administrator, where the alert includes information that the particular patient is requesting a modification to the current pain management schedule that exceeds one or more preset thresholds.

Once received via the administrator, method 700 may proceed to 735, where the administrator may approve the request via inputting information into the software application, or may deny the request. If, at 735, the request is approved, then method 700 may proceed to 745, where method 700 may include proceeding with providing the change to the pain management schedule as requested, in order to address the level of pain that the patient is experiencing. Similarly, returning to 725, under circumstances where the request does not exceed preset thresholds, then method 700 may proceed to 745 where the method includes proceeding with providing the change to the pain management schedule as requested.

Returning to 735, in a case where the request is approved, the approval may include temporarily overriding one or more thresholds such that the request may be allowed, but where in response to future requests, an alert may again be sent to the administrator such that requested changes to the schedule that exceed one or more thresholds consistently rely on administrator input. In this way, issues related to tolerance and/or dependency may be reduced.

At 735, in a case where the request is not approved, method 700 may proceed to 740. At 740, method 700 may include relying on manual input via the administrator to the software application in order to address the current level of pain that the patient is experiencing. In other words, rather than simply overriding one or more preset threshold to allow the change to the pain management schedule, active intervention on the part of the administrator may be conducted. The active intervention may include manual manipulation of one or more settings, parameters, thresholds, sequences of treatment, durations of particular treatments, etc., as determined by the administrator.

In response to any changes in pain management (see steps 730-740), or under circumstances where changes to the pain management as a function of the request are allowed without administrator intervention, method 700 may include, at 750, collecting data pertaining to the request and mitigating action (if any) taken to address the request. Said another way, at step 750 of method 700, data may be collected in similar fashion as that discussed with regard to 718, in order to allow for machine learning of optimal pain management strategy for particular patients.

Proceeding to 755, method 700 may include continuing to provide bioelectric neuromodulation and/or pharmacological treatments according to the updated schedule, the updated schedule a function of the request for the change in the way in which pain management is delivered to the patient. It may be understood that data may continue to be collected as discussed above with regard to steps 750 and 718 of method 700, while the current schedule is being provided. In a case where further changes to the currently provided pain management schedule are requested, method 700 may repeat.

Thus, discussed herein, a dual-action catheter system may comprise a catheter including a catheter lumen. The system may further include a bioelectric neuromodulation stylet of a diameter smaller than the lumen of the catheter for insertion of the bioelectric neuromodulation stylet within the catheter lumen. The system may further include one or more electrodes positioned at a tip end of the bioelectric neuromodulation stylet. The system may further include a delivery pathway for delivery of pharmacological treatments therethrough while the bioelectric neuromodulation stylet is inserted within the catheter lumen.

In such a system, the tip end of the bioelectric neuromodulation stylet may protrudes a predetermined distance from a distal end of the catheter when inserted within the catheter lumen.

In such a system, the delivery pathway may comprise a space between the bioelectric neuromodulation stylet and a wall of the catheter that defines the catheter lumen. In such an example, the bioelectric neuromodulation stylet may be solid.

In such a system, the delivery pathway may comprise a hollow portion of the bioelectric neuromodulation stylet.

In such a system, the system may further comprise a deployable stylet anchor positioned within a predetermined distance of a tip of the bioelectric neuromodulation stylet.

In such a system, the system may further comprise a a deployable catheter anchor positioned within a predetermined distance of a distal end of the catheter.

In such a system, the system may further comprise a catheter decoupler connector for selectively mechanically coupling the catheter to a decoupler.

In such a system, the system may further comprise a stylet decoupler connector for selectively mechanically coupling the bioelectric neuromodulation stylet to a decoupler.

In such a system, the bioelectric neuromodulation stylet may further comprise an electrical input source for receiving commands from an electrical stimulating source for delivering bioelectric neuromodulation via the one or more electrodes.

In such a system, the system may further comprise a pharmacological delivery connection for receiving the pharmacological treatments for delivery through the delivery pathway.

In such a system, the dual-action catheter system may further comprise a controller input connection for selectively communicably coupling a controller to the dual-action catheter system for controlling the one or more electrodes and for controlling the delivery of the pharmacological treatments.

In another example of the present disclosure, a pain management system for a patient comprises a dual-action catheter system comprising a catheter and a bioelectric neuromodulation stylet, the catheter including a lumen that receives the bioelectric neuromodulation stylet, and where the dual-action catheter system is delivers pharmacological treatments via a delivery pathway and further delivers bioelectric neuromodulation to the patient. The system may further include a controller for the dual-action catheter system. The system may further include a pain management application communicably coupled to the controller.

In such a system, the bioelectric neuromodulation stylet may include electrodes positioned at a tip end of the bioelectric neuromodulation stylet for delivering the bioelectric neuromodulation to the patient.

In such a system, the bioelectric neuromodulation stylet may include a stylet lumen, wherein the delivery pathway may comprise the stylet lumen. In such an example, the bioelectric neuromodulation stylet may further comprise a stylet anchor. In such an example, the delivery pathway may further comprise one or more passageways stemming from the stylet lumen for delivering the pharmacological treatments when the stylet anchor is deployed.

In such a system, the bioelectric neuromodulation stylet may be solid, where the delivery pathway may comprise a space between the bioelectric neuromodulation stylet and a wall defining the catheter lumen.

In such a system, the catheter may further include a deployable catheter anchor.

In such a system, the system may further comprise a decoupler selectively mechanically coupled to the catheter.

In such a system, the system may further comprise a decoupler selectively mechanically coupled to the bioelectric neuromodulation stylet.

In such a system, the pain management application may include options for controlling the delivering of the pharmacological treatments and bioelectric neuromodulation to the patient.

In such a system, the pain management application may be accessed by the patient or an administrator, and may include an option for inputting a current pain level experienced by the patient.

In such a system, the controller and the pain management application may be communicably coupled via a network to a server that stores data retrieved from the controller and the pain management application. In such an example, the system may further comprise an analytics module that executes a machine learning algorithm on the data stored at the server and returns output from the machine learning algorithm to the pain management application.

In yet another example of the present disclosure, a method for managing pain in a patient may comprise selectively delivering pharmacological treatments to the patient via a dual-action catheter system; selectively delivering bioelectric neuromodulation treatments to the patient via the dual-action catheter system; receiving a pain level input via the patient through a pain management application communicably coupled to a controller that is in turn communicable coupled to the dual-action catheter system; and controlling selectively delivering the pharmacological treatments and controlling selectively delivering the bioelectric neuromodulation treatments to the patient based on the received pain level.

In such a method, the dual-action catheter system may include a catheter with a lumen that receives a bioelectric neuromodulation stylet therethrough. In such an example, the pharmacological treatments and the bioelectric neuromodulation are selectively delivered under conditions where the bioelectric neuromodulation stylet is inserted into the lumen of the catheter. In one example, selectively delivering the pharmacological treatments may be via a lumen of the bioelectric neuromodulation stylet. In another example, selectively delivering the pharmacological treatments may be via a space between the bioelectric neuromodulation stylet and an inner wall of the catheter lumen. In yet another example, selectively delivering the bioelectric neuromodulation is via the bioelectric neuromodulation stylet.

In such a method, selectively delivering the bioelectric neuromodulation may occur at a same time as selectively delivering the pharmacological treatments.

In such a method, selectively delivering the bioelectric neuromodulation may occur at a different time than selectively delivering the pharmacological treatments.

In such a method, the method may further comprise setting thresholds associated with the managing of pain via the pain management application.

In such a method, the method may further comprise deploying a catheter anchor associated with the catheter prior to selectively delivering the pharmacological treatments and selectively delivering the bioelectric neuromodulation.

In such a method, the method may further comprise deploying a stylet anchor associated with the bioelectric neuromodulation stylet prior to selectively delivering the pharmacological treatments and selectively delivering the bioelectric neuromodulation.

In such a method, the method may further comprise mechanically coupling the catheter to a decoupler and securing the decoupler to a skin of the patient prior to selectively delivering the pharmacological treatments and selectively delivering the bioelectric neuromodulation.

In such a method, the method may further comprise mechanically coupling the bioelectric neuromodulation stylet to a decoupler and securing the decoupler to a skin of the patient prior to selectively delivering the pharmacological treatments and selectively delivering the bioelectric neuromodulation.

1. A dual-action catheter system, comprising:
a catheter including a catheter lumen;
a bioelectric neuromodulation stylet of a diameter smaller than the lumen of the catheter for insertion of the bioelectric neuromodulation stylet within the catheter lumen;
one or more electrodes positioned at a tip end of the bioelectric neuromodulation stylet; and
a delivery pathway for delivery of pharmacological treatments therethrough while the bioelectric neuromodulation stylet is inserted within the catheter lumen.

2. The dual-action catheter system of claim 1, wherein the tip end of the bioelectric neuromodulation stylet protrudes a predetermined distance from a distal end of the catheter when inserted within the catheter lumen.

3. The dual-action catheter system of claim 1, wherein the delivery pathway comprises a space between the bioelectric neuromodulation stylet and a wall of the catheter that defines the catheter lumen.

4. The dual-action catheter system of claim 3, wherein the bioelectric neuromodulation stylet is solid.

5. The dual-action catheter system of claim 1, wherein the delivery pathway is via a hollow portion of the bioelectric neuromodulation stylet.

6. The dual-action catheter system of claim 1, further comprising a deployable stylet anchor positioned within a predetermined distance of a tip of the bioelectric neuromodulation stylet.

7. The dual-action catheter system of claim 1, further comprising a deployable catheter anchor positioned within a predetermined distance of a distal end of the catheter.

8. The dual-action catheter system of claim 1, further comprising a catheter decoupler connector for selectively mechanically coupling the catheter to a decoupler.

9. The dual-action catheter system of claim 1, further comprising a stylet decoupler connector for selectively mechanically coupling the bioelectric neuromodulation stylet to a decoupler.

10. The dual-action catheter system of claim 1, further comprising a pharmacological delivery connection for receiving the pharmacological treatments for delivery through the delivery pathway.

11. A pain management system for a patient, comprising:
a dual-action catheter system comprising a catheter and a bioelectric neuromodulation stylet, the catheter including a lumen that receives the bioelectric neuromodulation stylet, and where the dual-action catheter system delivers pharmacological treatments via a delivery pathway and further delivers bioelectric neuromodulation to the patient;
a controller for the dual-action catheter system; and
a pain management application communicably coupled to the controller.

12. The pain management system of claim 11, wherein the bioelectric neuromodulation stylet includes electrodes positioned at a tip end of the bioelectric neuromodulation stylet for delivering the bioelectric neuromodulation to the patient.

13. The pain management system of claim 11, wherein the bioelectric neuromodulation stylet includes a stylet lumen, and wherein the delivery pathway comprises the stylet lumen.

14. The pain management system of claim 11, wherein the bioelectric neuromodulation stylet is solid; and
wherein the delivery pathway comprises a space between the bioelectric neuromodulation stylet and a wall defining the lumen of the catheter.

15. The pain management system of claim 11, wherein the pain management application includes options for controlling the delivering of the pharmacological treatments and bioelectric neuromodulation to the patient.

16. A method for management of pain in a patient, comprising:
selectively delivering pharmacological treatments to the patient via a dual-action catheter system;
selectively delivering bioelectric neuromodulation treatments to the patient via the dual-action catheter system;
receiving a pain level input via the patient through a pain management application communicably coupled to a controller that is in turn communicably coupled to the dual-action catheter system; and
controlling selectively delivering the pharmacological treatments and controlling selectively delivering the bioelectric neuromodulation treatments to the patient based on the received pain level.

17. The method of claim 16, where the dual-action catheter system includes a catheter with a lumen that receives a bioelectric neuromodulation stylet therethrough; and
wherein the pharmacological treatments and the bioelectric neuromodulation are selectively delivered under conditions where the bioelectric neuromodulation stylet is inserted into the lumen of the catheter.

18. The method of claim 17, wherein selectively delivering the pharmacological treatments is via a bioelectric neuromodulation stylet lumen.

19. The method of claim 17, wherein selectively delivering the pharmacological treatments is via a space between the bioelectric neuromodulation stylet and an inner wall of the catheter.

20. The method of claim 17, further comprising deploying a catheter anchor associated with the catheter prior to selectively delivering the pharmacological treatments and selectively delivering the bioelectric neuromodulation.
